(19) United States
(12) Patent Application Publication
(10) Pub. No.: US 2006/0265035 A1
(43) Pub. Date: Nov. 23, 2006

(54) Medical treatment instrument, water supply / suction system for medical treatment instrument

(75) Inventors: Chie Yachi, Tokyo (JP); Masaru Imoto, Tokyo (JP); Shinya Masuda, Tokyo (JP); Ryoji Sakai, Tokyo (JP); Eiji Murakami, Tokyo (JP)

Correspondence Address: Thomas Spinelli;Scully, Scott, Murphy & Presser, Suite 300, 400 Garden City Plaza, Garden City, NY, 11530, US

(73) Assignee: OLYMPUS MEDICAL SYSTEMS CORP., TOKYO (JP)

(21) Appl. No.: 11/433,982

(22) Filed: May 15, 2006

(30) Foreign Application Priority Data
May 13, 2005 (JP) 2005-141536
Nov. 18, 2005 (JP) 2005-334665

(51) Int. Cl. A61F 2/00

(52) U.S. Cl. 607/101; 606/41

(57) ABSTRACT

A medical treatment instrument of the present invention includes an elongated insertion portion inserted into a body cavity, a treatment portion provided at a tip end of the insertion portion for treating a tissue in the body cavity by heating, a cylindrical member into which a part of the insertion portion is inserted, a flow passage formed between an outer circumference of the insertion portion and an inner circumference of the cylindrical member and through which one of a fluid supplied to the treatment portion and the fluid sucked from within the body cavity flows, an inlet/outlet communicating with the flow passage for performing one of supply of the fluid supplied to the treatment portion and discharge of the fluid sucked from within the body cavity, and an opening portion communicating with the flow passage and opened opposite to the treatment portion.

This application claims benefit of Japanese Applications No. 2005-141536 filed in Japan on May 13, 2005 and No. 2005-334665 filed in Japan on Nov. 18, 2005 the contents of which are incorporated by this reference.

### BACKGROUND OF THE INVENTION

1. Field of the Invention

The present invention relates to a medical treatment instrument, a water supply/suction system for a medical treatment instrument in which a treatment portion for treating a tissue to be treated by heating the tissue to be treated in a body cavity is provided to an elongated insertion portion.

2. Description of the Related Art

Various ultrasonic treatment instruments have been proposed as a medical treatment instrument for ablating a tissue or removing an organic substance or any other accumulation using ultrasonic vibration.

European Patent No. 0645987, for example, discloses an ultrasonic treatment instrument capable of ultrasonic treatment of a tissue to be treated and suction of particles such as blood in a body cavity generated in the ultrasonic treatment (hereinafter referred to as aerosol) using ultrasonic vibration.

Specifically, the ultrasonic treatment instrument disclosed in European Patent No. 0645987 has an ultrasonic vibrator (hereinafter referred to simply as vibrator) to a hand piece, and has such a construction that the vibrator is connected to an elongated probe for ultrasonic transmission (hereinafter referred to simply as probe) provided with a treatment portion for ultrasonic treatment of a tissue to be treated. From this, ultrasonic vibration generated from the vibrator by driving is transmitted to the treatment portion through the probe.

Also, the ultrasonic treatment instrument has a sheath circumferentially covering a position opposite to a treatment position of a probe (hereinafter referred as a tip end side) so that an annular open space is formed between it and an outer circumference of the probe tip end side.

The sheath has a suction hole formed to communicate to the open space, and to the suction hole, a suction source is connected through a suction tube. Moreover, in the ultrasonic treatment instrument, a pipeline connected to a water supply source for spraying water to the treatment portion is disposed while being opened on the tip end side.

The ultrasonic treatment instrument which is constructed as above and disclosed in European Patent No. 0645987 performs ultrasonic treatment of a tissue to be treated using an aerosol or mist generated by cavitation by transmitting the ultrasonic vibration to the treatment portion and by spraying water to the probe. Also, the aerosol generated during the treatment can be sucked by the suction source through the opening, the suction hole and the suction tube formed in the sheath.

### SUMMARY OF THE INVENTION

Briefly, a medical treatment instrument according to the present invention comprises an elongated insertion portion to be inserted into a body cavity, a treatment portion provided at a tip end of the insertion portion for heating the tissue to be treated in the body cavity for treatment, a cylindrical member into which at least a part of the insertion portion is inserted, a flow passage formed between an outer circumference of the insertion portion and an inner circumference of the cylindrical member and through which at least one of a fluid supplied to the treatment portion and a fluid sucked from within the body cavity flows, a flow port communicating with the flow passage for performing at least either of supply to the flow passage of the fluid to be supplied to the treatment portion and discharge from the flow passage of the fluid sucked from inside the body cavity, and an opening communicating with the flow passage and opened opposite to the treatment portion.

The above and other objects, features and advantages of the invention will become more clearly understood from the following description referring to the accompanying drawings.

### BRIEF DESCRIPTION OF THE DRAWINGS

FIG. 1 is a view of an ultrasonic treatment instrument with only a part of an insertion portion in section showing a first embodiment of the present invention;

FIG. 2 is an enlarged sectional view of a base end side of the insertion portion in FIG. 1;

FIG. 3 is a view showing a variation in which a gripping groove is provided on an outer circumference of a connection screw in FIG. 2;

FIG. 4 is an enlarged view of a tip end side of the insertion portion in FIG. 1;

FIG. 5 is a sectional view along V-V line in FIG. 4;

FIG. 6 is a block diagram schematically showing a construction of an ultrasonic treatment apparatus having the ultrasonic treatment instrument in FIG. 1;

FIG. 7 is an exploded view removing a sheath 9 from an insertion portion 4 of an ultrasonic treatment instrument 1 in FIG. 1;

FIG. 8 is a flowchart showing a control method of an ultrasonic treatment apparatus when treating a tissue to be treated in a body cavity using the ultrasonic treatment instrument in FIG. 1;

FIG. 9 is a view showing a state where the tip end side of the insertion portion of the sheath in FIG. 1 is moved to a position covering the tip end side of a gripping portion;

FIG. 10 is a view of an ultrasonic treatment instrument with only a part of an insertion portion in section showing a second embodiment of the present invention;

FIG. 11 is an enlarged sectional view of the base end side of the insertion portion in FIG. 10;

FIG. 12 is an enlarged sectional view of the tip end side of the insertion portion in FIG. 10;

FIG. 13 is a view of an ultrasonic treatment instrument with only a part of an insertion portion in section showing a third embodiment of the present invention;

FIG. 14 is a sectional view along XIV-XIV line in FIG. 13;

FIG. 15 is an enlarged sectional view of the base end side of the insertion portion in FIG. 13;

FIG. 16 is an enlarged sectional view of the tip end side of the insertion portion in FIG. 13;

FIG. 17 is a sectional view along XVII-XVII line in FIG. 16:

FIG. 18 is a partial sectional view showing a variation providing a groove at a position opposite to a grip portion of a probe of the ultrasonic treatment instrument in FIGS. 1, 10 and 13;

FIG. 19 is a partial sectional view showing a variation in which a single port into which a water/air supply port and a suction port are made common is provided to the ultrasonic treatment instrument in FIG. 1;

FIG. 20 is an enlarged sectional view of the base end side of the insertion portion in FIG. 19;

FIG. 21 is a block diagram schematically showing a construction of an ultrasonic treatment apparatus having the ultrasonic treatment instrument in FIG. 19;

FIG. 22 is a block diagram schematically showing another construction of the ultrasonic treatment apparatus in FIG. 21;

FIG. 23 is a view of an ultrasonic treatment instrument with only a part of an insertion portion in section showing a fourth embodiment of the present invention;

FIG. 24 is an enlarged view of the tip end side of the insertion portion in FIG. 23;

FIG. 25 is a view showing a state where a slider member in FIG. 24 is moved to the tip end in an axial direction of insertion;

FIG. 26 is a sectional view along IIXVI-IIXVI/line in FIG. 25;

FIG. 27 is a view of an ultrasonic treatment instrument with only a part of an insertion portion in section showing a fifth embodiment of the present invention;

FIG. 28 is an enlarged sectional view of the base end side of the insertion portion in FIG. 27;

FIG. 29 is a sectional view along IIXIX-IIXIX line in FIG. 28;

FIG. 30 is an enlarged sectional view of the tip end side of the insertion portion in FIG. 27;

FIG. 31 is a sectional view along IIIXI-IIIXI line in FIG. 30;

FIG. 32 is a block diagram schematically showing a construction of an ultrasonic treatment apparatus having the ultrasonic treatment instrument in FIG. 27;

FIG. 33 is a flowchart showing a control method of an ultrasonic treatment apparatus when treating a tissue to be treated in a body cavity using the ultrasonic treatment instrument in FIG. 27;

FIG. 34 is a flowchart showing a control method of an ultrasonic treatment apparatus having an ultrasonic treatment instrument showing a sixth embodiment of the present invention;

FIG. 35 is an enlarged sectional view of a tip end side of a sheath of an ultrasonic treatment instrument showing a seventh embodiment of the present invention;

FIG. 36 is a sectional view along IIIXVI-IIIXVI line in FIG. 35;

FIG. 37 is an enlarged sectional view of a tip end side of an ultrasonic treatment instrument showing an eighth embodiment of the present invention;

FIG. 38 is a sectional view along IIIXVIII-IIIXVIII line in FIG. 37;

FIG. 39 is a sectional view along IIIXIX-IIIXIX line in FIG. 37;

FIG. 40 is a block diagram schematically showing a construction of an ultrasonic treatment apparatus having an ultrasonic treatment instrument showing a ninth embodiment of the present invention;

FIG. 41 is a flowchart showing a control method of an ultrasonic treatment apparatus in FIG. 40;

FIG. 42 is an enlarged sectional view of a base end side of an insertion portion of an ultrasonic treatment instrument showing a tenth embodiment of the present invention;

FIG. 43 is a sectional view along IVXIII-IVXIII line in FIG. 42; and

FIG. 44 is a view showing an ultrasonic treatment instrument with only a part of an insertion portion in section showing an eleventh embodiment of the present invention.

### DETAILED DESCRIPTION OF PREFERRED EMBODIMENTS

Embodiments of the present invention will be described below with reference to the attached drawings.

Medical treatment instruments for various treatments such as surgeries using heat include ultrasonic treatment instruments using ultrasonic wave, high-frequency treatment instrument using Joule heat or the like by high-frequency electric current, laser treatment instrument using laser beam, RF treatment instruments using electromagnetic wave, etc., but an ultrasonic treatment instrument for coagulation ablation is used as an example in these embodiments.

### First Embodiment

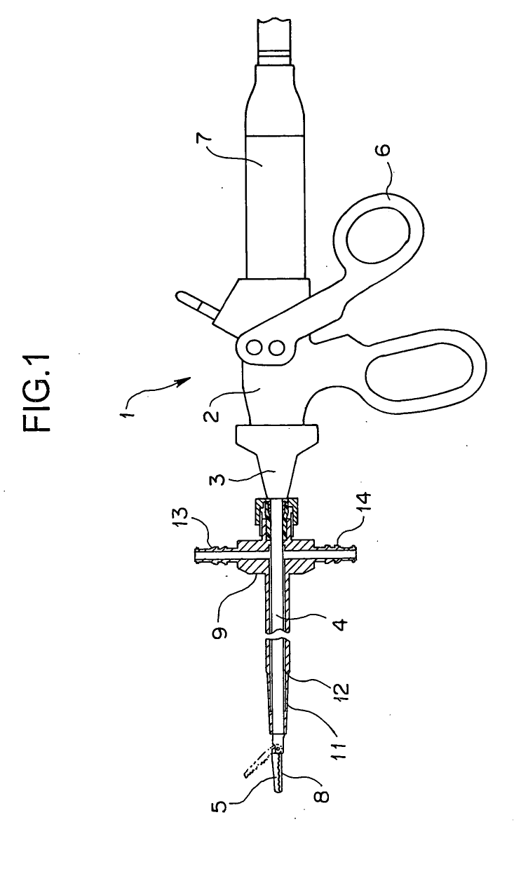

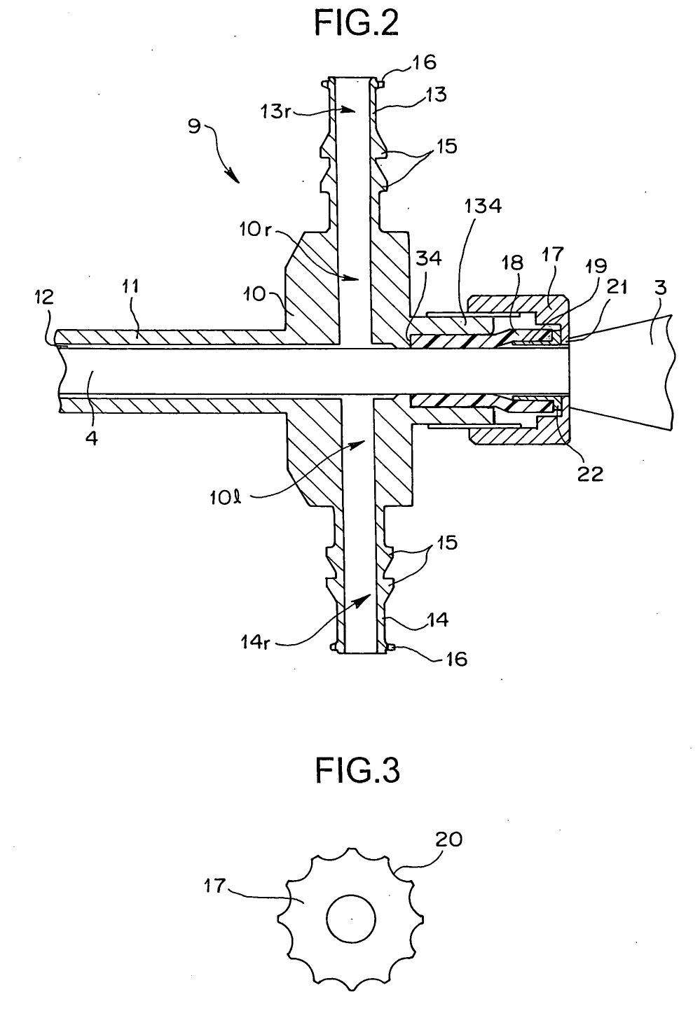

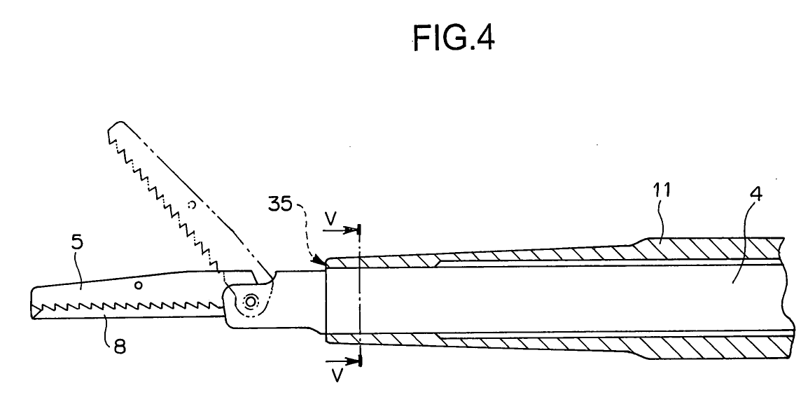

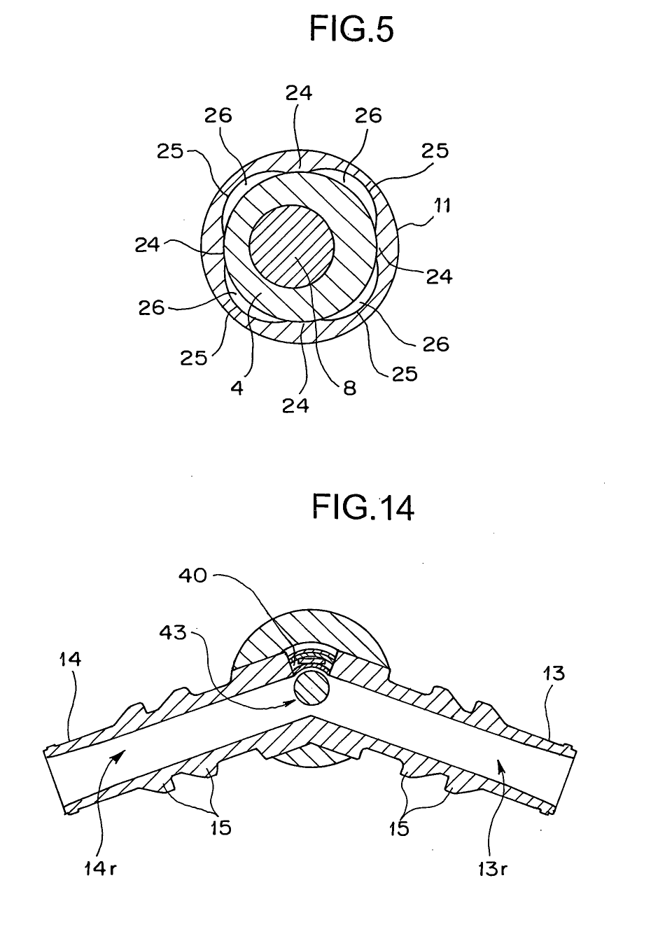

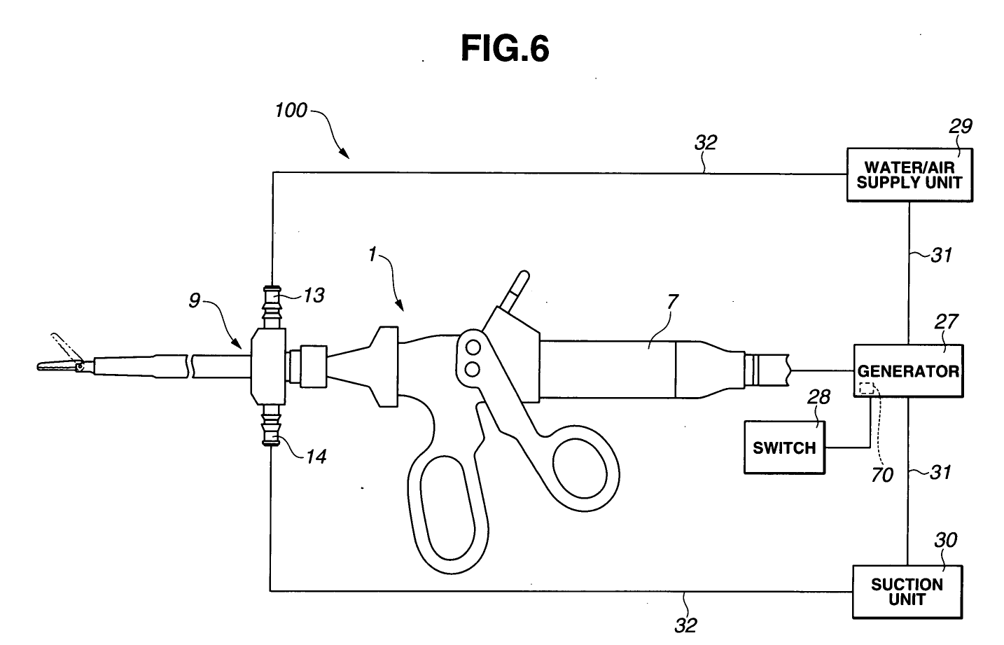

FIG. 1 is a view of an ultrasonic treatment instrument with only a part of an insertion portion in section showing a first embodiment of the present invention, FIG. 2 is an enlarged sectional view of a base end side of the insertion portion in FIG. 1, FIG. 3 is a view showing a variation in which a gripping groove is provided on an outer circumference of a connection screw in FIG. 2, FIG. 4 is an enlarged view of a tip end side of the insertion portion in FIG. 1, FIG. 5 is a sectional view along V-V line in FIG. 4, and FIG. 6 is a block diagram schematically showing a construction of an ultrasonic treatment apparatus having the ultrasonic treatment instrument in FIG. 1.

In the description below, a side of the ultrasonic treatment instrument to be inserted into a body cavity is referred to as a tip end side and an operation portion side as a base end side.

As shown in FIG. 1, an ultrasonic treatment instrument 1 comprises an elongated insertion portion 4 to be inserted into a body cavity, and a grip portion 5, which is a treatment portion, is disposed at the tip end side of the insertion portion 4. The grip portion 5 constitutes an opening/closing portion comprised by a pair of jaws capable of opening/closing, for example, and a tissue to be treated in the body cavity is gripped and heated by the jaws for treatment of coagulation ablation or the like. The grip portion 5 is capable of opening/closing with respect to the tip end side of a probe 8, which will be described later.

Moreover, the base end side of the insertion portion 4 is connected to the tip end of a rotating knob 3, which is an operation portion. The rotating knob 3 is connected to the tip end of a fixed handle 2, which is the operation portion, for rotating operation of the insertion portion 4 in a direction around an axis of the insertion portion 4.

Moreover, a movable handle 6 for opening/closing operation of the pair of jaws of the grip portion 5 is rotatably mounted on the fixed handle 2 through a support shaft. Also, at the fixed handle 2, the elongated probe 8, which is a heating portion, inserted into the insertion portion 4 and having a tip end connected to the grip portion 5 and a base end connected to a vibrator 7 is mounted.

It is to be noted that the probe 8 is for various treatments such as coagulation for a tissue to be treated using heat generated by an electric power supplied from a generator 27 (See FIG. 6), which will be described later, and a portion connected to the grip portion 5 at the tip end side of the probe 8 constitutes a treatment portion.

On an outer circumference of the insertion portion 4, specifically, on an outer circumference excluding the tip end sides of the grip portion 5 of the insertion portion 4 and the probe 8, a water supply/suction sheath, which is a cylindrical member (hereinafter referred to simply as a sheath) 9 is attached. That is, the tip end sides of the grip portion 5 and the probe 8 are projected from the sheath 9. The sheath 9 is detachably attached to at least a part of the outer circumference of the insertion portion 4. That is, at least a part of the insertion portion 4 is inserted into the sheath 9.

As shown in FIG. 2, the sheath 9 has a major part comprising, for example, a flange-state body portion 10, a insertion portion 11 extending to the tip end side from substantially the center of the body portion 10 and having a diameter smaller than that of the body portion 10 and a connection portion 134 extending to the base end side from substantially the center of the body portion 10 and having a diameter smaller than that of the body portion 10. On an outer circumference of the connection portion 134, a screw is formed.

An inner diameter of a hole of the insertion portion 11 is formed larger than an outer diameter of the insertion portion 4. By this, a flow passage 12 is formed between an inner circumference of the insertion portion 11 and an outer circumference of the insertion portion 4. Also, flow passages 10r, 101 substantially perpendicular to a hole communicating from the tip end to the base end of the body portion 10 are formed. The flow passages 10r, 101 communicate to the flow passage 12.

Moreover, a water/air supply port 13 as an inlet for connection to a water/air supply unit 29 (See FIG. 6) having a pump for supplying water/air, which will be described later, is provided to the body portion 10 so that it is substantially perpendicular to the insertion portion 11. The water/air supply port 13 has a flow passage 13r, and the flow passage 13r communicates to the flow passage 10r of the body portion 10. That is, the flow passage 13r also communicates to the flow passage 12.

Moreover, a suction port 14, which is an outlet for connection to a suction unit 30 (See FIG. 6) having a pump for suction, which will be described later, is provided to the portion 10 so that it is substantially perpendicular to the insertion portion 11. The suction port 14 has a flow passage 14r, and the flow passage 14r communicates to a flow passage 101 of the body portion 10. That is, the flow passage 14r also communicates to the flow passage 12.

It is to be noted that the water/air supply port 13 and the suction port 14 may be provided on the body portion 10 with a free mounting angle, not limited to substantially perpendicular to the insertion portion 11, as long as it is in a range not interfering with the ultrasonic treatment instrument 1.

Also, the water/air supply port 13 and the suction port 14 constitutes a port performing at least one of supply of a fluid to the flow passage 12 to be supplied to the treatment portion comprised by the tip end sides of the grip portion 5 and the probe 8 or discharge from the flow passage 12 of a fluid sucked from inside the body cavity.

An outer circumference of the water/air supply port 13 is covered by one end of a tube 32 connecting the water/air supply port 13 to the water/air supply unit 29. Also, an outer circumference of the suction port 14 is covered by one end of the tube 32 connecting the suction port 14 to the suction unit 30.

On the outer circumferences of the water/air supply port 13 and the suction port 14, hook-state projection portions 15 are provided for preventing removal of the covering tube 32. By this, one ends of the tube 32 can be directly connected to each of the ports 13, 14.

Also, at an end on the outer circumference of the water/air supply port 13, the suction port 14, a luer-lock base to be connected to a luer-lock connector, not shown, connected to each of one ends of the tube 32 is provided, respectively. By this, the tube 32 with the luer-lock connector connected to one end can be connected to each of the ports 13, 14.

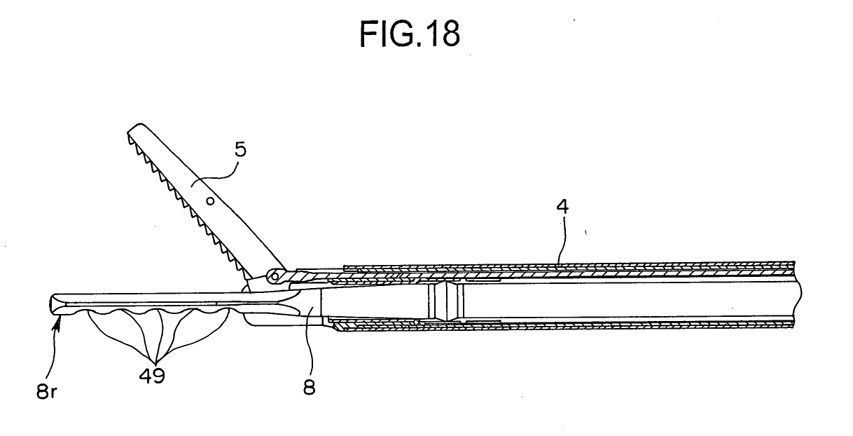

It is to be noted that only either one of the projection portion 15 or the luer-lock base 16 may be provided to each of the ports 13, 14. Also, the number of ports provided to the body portion 10 is not limited to 2, but may be arbitrary as long as it is 1 or more. The case of only 1 will be described below in detail referring to FIGS. 18 to 22.

An annular connection screw 17 is screwed with a thread portion formed on the outer circumference of the connection portion 134 of the body portion 10. The connection screw 17 is mounted on the outer circumference of the connection portion 134 holding an elastic member 18 and a sleeve 19 between a base end portion 21 of the connection screw 17 and a bottom portion 34 of the connection portion 134 in an axial direction of the insertion portion (hereinafter referred to as insertion axial direction).

The elastic member 18 and the sleeve 19 are formed in the annular state, and an inner circumference of each of the elastic member 18 and the sleeve 19 is formed larger than the outer diameter of the insertion portion 4 so that the insertion portion 4 can be inserted.

Moreover, an inner circumference of an opening formed on the base end portion 21 of the connection screw 17 is also formed larger than the outer diameter of the insertion portion 4 so that the insertion portion 4 can be inserted. It is to be noted that the inner circumference of the opening formed on the base end portion 21 is formed smaller than a diameter of the tip end side of the rotating knob 3.

Also, a grip groove 20 may be formed on an outer circumference of the connection screw 17 as shown in FIG. 3 so as to facilitate mounting on the connection portion 134.

In order to mount the connection screw 17, the elastic member 18 and the sleeve 19 constituted as above on the connection portion 134 of the body portion 10, first, the base end side of the elastic member 18 is inserted into the tip end side of the sleeve 19 and then, the base end portion of the elastic member 18 is made to abut against a flange 22 formed at the base end of the sleeve 19.

Next, in the state where the elastic member 18 and the sleeve 19 are combined together, the tip end side of the elastic member 18 is fitted with the inner circumference of the connection portion 134 of the body portion 10 and finally, the connection screw 17 is screwed with the outer circumference of the connection portion 134 as if the elastic member 18 and the sleeve 19 are crushed in the insertion axial direction by the base end portion 21 of the connection screw 17.

At this time, the length of the elastic member 18 and the sleeve 19 in combination is shorter than the length of the hole at the base end portion of the connection portion 134 plus the length of the hole of the connection screw 17.

As shown in FIG. 4, the tip end of the sheath 9 when the sheath 9 is attached to the insertion portion 4 is located on the base end side from the grip portion 5 so that the tip end sides of the grip portion 5 and the probe 8 are exposed. The tip end of the insertion portion 11 of the sheath 9 is formed in the tapered state.

As shown in FIG. 5, four, for example, projecting state support portions 24 in contact with the outer circumference of the insertion portion 4 are formed on the inner circumference of the tip end portion of the insertion portion 11, and four arcs 25 are formed on the inner circumference of the tip end portion of the insertion portion 11, for connecting the four support portions 24 along the inner circumference of the tip end portion of the insertion portion 11.

Tip end flow passages 26 are formed between the four arcs 25 divided by the four support portions 24 and the outer circumference of the insertion portion 4, and the tip end flow passages 26 communicate to the flow passage 12 formed between the inner circumference of the insertion portion 11 and the outer circumference of the insertion portion 4.

Also, the tip end flow passage 26 is opened as an opening portion 35 at the base end side of the grip portion 5 in the vicinity of a position opposite to the grip portion 5 or the probe 8 due to location of the tip end of the sheath 9 when the sheath 9 is attached to the insertion portion 4 at the base end side from the grip portion 5. The opening portion 35 is provided at a position in the vicinity of the tip end side of the probe 8 from the grip portion 5. That is, the opening portion 35 is opened at least toward the tip end side of the probe 8.

As shown in FIG. 6, an ultrasonic treatment apparatus 100 has its major part comprised by the ultrasonic treatment instrument 1, a generator 27, a switch 28, the water/air supply unit 29, which is a supply source, and a suction unit 30, which is a suction source.

The vibrator 7 of the ultrasonic treatment instrument 1 is connected to the generator 27 for performing ultrasonic driving. Also, the switch 28 for oscillating the vibrator 7 is connected to the generator 27.

The generator 27, the water/air supply unit 29 and the suction unit 30 are electrically connected by a cable 31 so that they can be operated in the interlock manner. Moreover, the water/air supply unit 29 is connected to the water/air supply port 13 of the ultrasonic treatment instrument 1 by the tube 32, and the suction unit 30 is connected to the suction port 14 of the ultrasonic treatment instrument 1 by the tube 32.

Here, when a fluid of water or air is supplied from the water/air supply unit 29, on one hand, the fluid advances into the flow passage 13r of the water/air supply port 13 through the tube 32 connected to the water/air supply unit 29, passes through the flow passage 10r, the flow passage 12 between the inner circumference of the insertion portion 11 of the sheath 9 and the outer circumference of the insertion portion 4, and is supplied from the tip end opening portion 35 of the tip end flow passage 26 to the tip end sides of the grip portion 5 and the probe 8.

On the other hand, when the fluid such as smoke, mist, etc. is sucked by the suction unit 30 from the tip end opening portion 35, the fluid advances from the tip end opening portion 35 to the tip end flow passage 26, passes through the flow passages 12, 101, 14r and is discharged from the suction unit 30 through the tube 32 connected to the suction port 14.

Next, operation of the so-constituted ultrasonic treatment instrumental of the embodiment will be described below using FIGS. 1 to 6 and FIGS. 7 to 9.

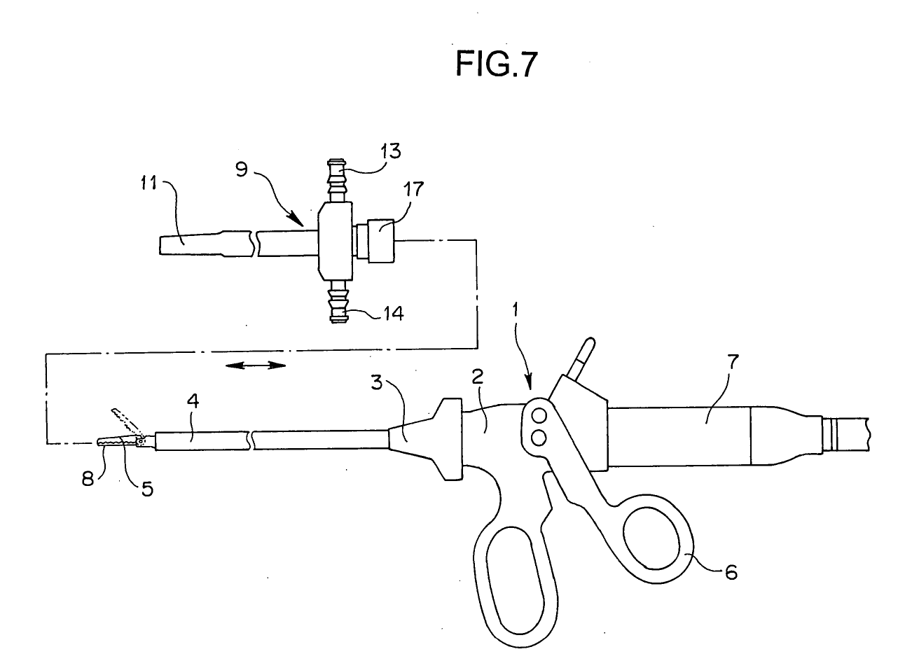

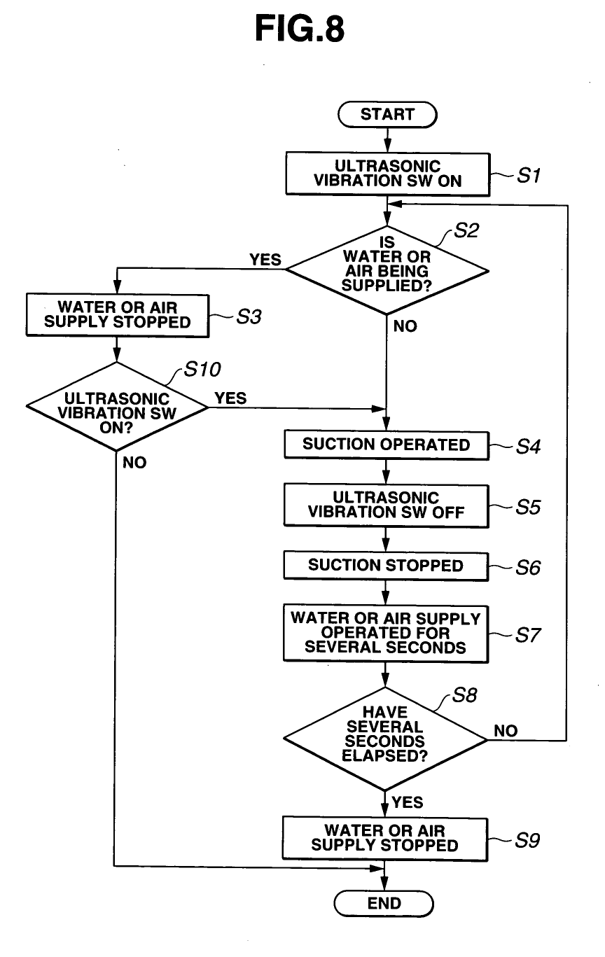

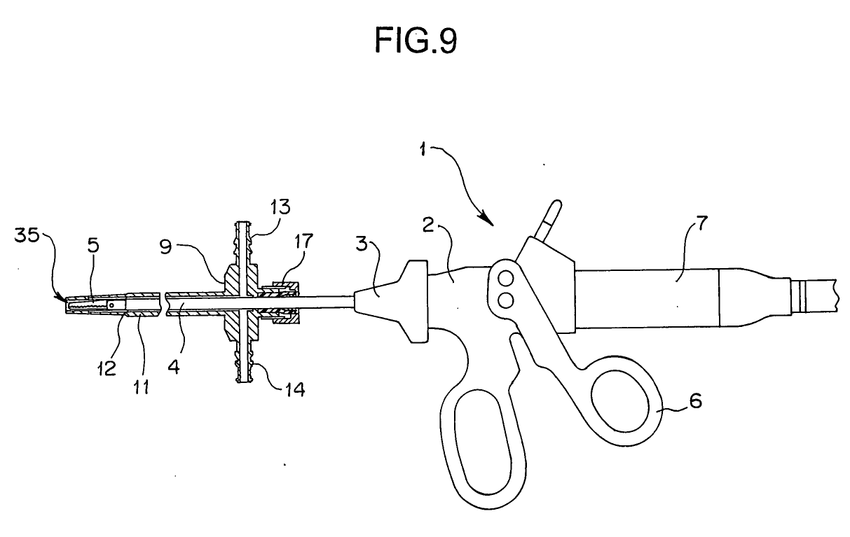

FIG. 7 is an exploded view with a sheath 9 removed from the insertion portion 4 of the ultrasonic treatment instrument 1 in FIG. 1, FIG. 8 is a flowchart showing a control method of an ultrasonic treatment apparatus when treating a tissue to be treated in a body cavity using the ultrasonic treatment instrument in FIG. 1, and FIG. 9 is a view showing a state where the tip end side of the insertion portion of the sheath in FIG. 1 is moved to a position covering the tip end side of a gripping portion.

First, as shown in FIG. 7, the sheath 9 is inserted to the insertion portion 4 of the ultrasonic treatment instrument 1 from the base end side of the sheath 9 with respect to the tip end side of the insertion portion 4 till it abuts against the rotating knob 3 and then, as shown in FIG. 2, the connection screw 17 is screwed to the outer circumference of the connection portion 134 as if the elastic member 18 and the sleeve 19 are crushed in the insertion axial direction by the base end portion 21 of the connection portion 17.

As a result, the inner diameter of the elastic member 18 is made smaller, and the inner circumference of the elastic member 18 is brought into close contact with the outer circumference of the insertion portion 4. By this, the sheath 9 is fixed to the ultrasonic treatment instrument 1. At this time, as mentioned above, the flow passage 12 is formed between the outer circumference of the insertion portion 4 and the inner circumference of the insertion portion 11. The flow passage 12 communicates to the tip end flow passage 26, the flow passages 10r, 101 of the body portion 10, the flow passage 13r of the water/air supply port 13 and the flow passage 14r of the suction port 14.

After that, the water/air supply port 13 is connected to the water/air supply unit 29 by the tube 32 and moreover, the suction port 14 is connected to the suction unit 30 by the tube 32 and then, the insertion portion 4 of the ultrasonic treatment instrument 1 is inserted into the body cavity so as to treat a tissue to be treated in the body cavity.

The operation below is performed by a control signal sent from control means 70, which is a control portion disposed in the generator 27 of the ultrasonic treatment apparatus 100 to the water/air supply unit 29 and the suction unit 30 through a communication line, not shown. That is, the operation is performed by operation control of the control means 70.

In treating the tissue to be treated in the body cavity, first, the tissue to be treated is gripped by the grip portion 5 and then, as shown in FIG. 8 at Step S1, when the switch 28 (See FIG. 6) to be an ultrasonic vibration switch (SW) is turned on, the vibrator 7 receives supply of electric power from the generator 27 and performs ultrasonic driving. The vibration of the vibrator 7 is transmitted to the grip portion 5 through the probe 8. By this, the tissue to be treated is heated by friction with the tissue to be treated by vibration of the probe 8 on the tip end side, and treatment such as coagulation ablation is performed.

Next, at Step S2, it is determined whether a fluid of water or air is supplied from the opening portion 35 to the grip portion 5 or the probe 8 by operation of the water/air supply unit 29.

On one hand, if the fluid of air or water is being supplied from the opening portion 35, the routine branches to Step S3, where supply of the fluid of water or air from the opening portion 35 is stopped by stopping the water/air supply unit 29 and after it is confirmed again at Step S10 that the switch 28 is on, the routine moves onto Step S4. On the other hand, if a fluid of water or air is not supplied from the opening portion 35, the routine goes onto Step S4.

At Step S4, in conjunction with heating treatment of the tissue to be treated by the grip portion 5, the suction unit 30 is operated, and mist or smoke generated in the treatment is sucked. Specifically, the fluid sucked by the suction unit 30 from the tip end opening portion 35 advances from the tip end opening portion 35 into the tip end flow passage 26, flows through the flow passages 12, 101, 14r and is discharged by the suction unit 30 through the tube 32 connected to the suction port 14.

At this time, as shown in FIG. 9, since the sheath 9 is connected to the elastic member 18, it is capable of sliding on the insertion portion 4. Therefore, the fluid may be sucked from the tip end of the insertion portion 4 after sliding the insertion portion 11 of the sheath portion 9 toward the tip end side so as to cover the grip portion 5 and then, by pushing out the tip end opening portion 35 to the tip end side from the grip portion 5.

When the treatment by the grip portion 5 of the tissue to be treated is finished, at Step S5, the switch 28 (See FIG. 6) is turned off, power supply from the generator 27 is shut down and the vibrator 7 is stopped.

Then, in conjunction with the stop of the vibrator 7, at Step S6, the suction unit 30 is stopped and suction of the mist or smoke generated in the treatment is stopped. After that, the routine goes onto Step S7.

At Step S7, when the water/air supply unit 29 is operated for preset several seconds, a fluid of water or air is supplied from the tip end opening portion 35 to the grip portion 5 or the probe 8.

Specifically, the fluid supplied from the water/air supply unit 29 advances into the flow passage 13r of the water/air supply port 13 through the tube 32 and then, flows through the flow passage 12 between the inner circumference of the insertion portion 11 of the sheath 9 and the outer circumference of the insertion portion 4 and is supplied from the tip end opening portion 35 of the tip end flow passage 26 to the grip portion 5 or the probe 8. By this, the grip portion 5 or the probe 8 which has been heated by the treatment is cooled. Also, stain of the grip portion 5 having adhered due to the treatment can be removed by supply of a fluid of water or air.

Next, at Step S8, it is determined whether the time for supply of the fluid of water or air from the tip end portion 35 has reached the preset several seconds or not. If the preset several seconds have not elapsed, the routine returns to Step S2, and at Step S3, after the supply time of the fluid of water or air has passed for several seconds, the water/air supply unit 29 is stopped and at the subsequent Step S10, the routine is finished after the off state of the switch 28 is confirmed.

Returning to Step S8, if the time during which the fluid of air or water is supplied from the tip end opening portion 35 has reached the preset several seconds, the routine moves onto Step S9 and finally at Step S9, the water/air supply unit 29 is stopped so as to stop supplying the fluid of water or air from the tip end opening portion 35 and then, the routine is finished.

In this way, in the ultrasonic treatment instrument 1 of this embodiment, the flow passages 10r, 12, 13r, 26 for supplying a fluid of water or air to the ultrasonic treatment instrument 1 are provided and the flow passages 101, 12, 14r, 26 for sucking the fluid from within the body cavity in a treatment are provided. That is, the flow passage 12 provided between the inserting portion 4 and the insertion portion 11 is a flow passage for supplying a fluid of water or air and also serves as a flow passage for sucking the fluid from within the body cavity.

Also, the opening portion 35 of the flow passages 10r, 101, 12, 13r, 14r, 26 is provided so that it is located opposite to the vicinity of the grip portion 5 and the probe 8, at the tip end side of the ultrasonic treatment instrument 1, for example.

By this, since the smoke or mist generated in an ultrasonic treatment can be effectively sucked by the suction unit 30 through the flow passages 101, 12, 14r, 26, the treatment using the treatment instrument can be surely performed while keeping the visual field of an operator all the time.

Also, since the fluid of water or air can be supplied by the water/air supply unit 29 from the opening portion 35 to the grip portion 5 and the probe 8 whose temperature has become high due to frictional heat through the flow passages 10r, 12, 13r, 26 immediately after an ultrasonic treatment, the heated grip portion 5 or the probe 8 can be rapidly cooled after the treatment.

Moreover, it is not necessary any more to provide two flow passages of a flow passage for supplying a fluid of water or air to the insertion portion 4 and a flow passage for sucking the fluid from within the body cavity.

From the above, an ultrasonic treatment instrument whose convenience in use is improved for operators can be provided.

A variation will be described below.

In this embodiment, it is shown that the fluid of water or air supplied from the opening portion 35 is used for cooling the grip portion 5 and the probe 8 after a treatment. Not limited to this, the fluid of water or air may be used for washing inside the body cavity by being supplied into the body cavity.

By this, insertion of a water supply/suction pipe from another port of the ultrasonic treatment instrument 1 can be omitted and moreover, a labor of inserting only the water supply/suction pipe after removing the ultrasonic treatment instrument from within the body cavity can be omitted.

### Second Embodiment

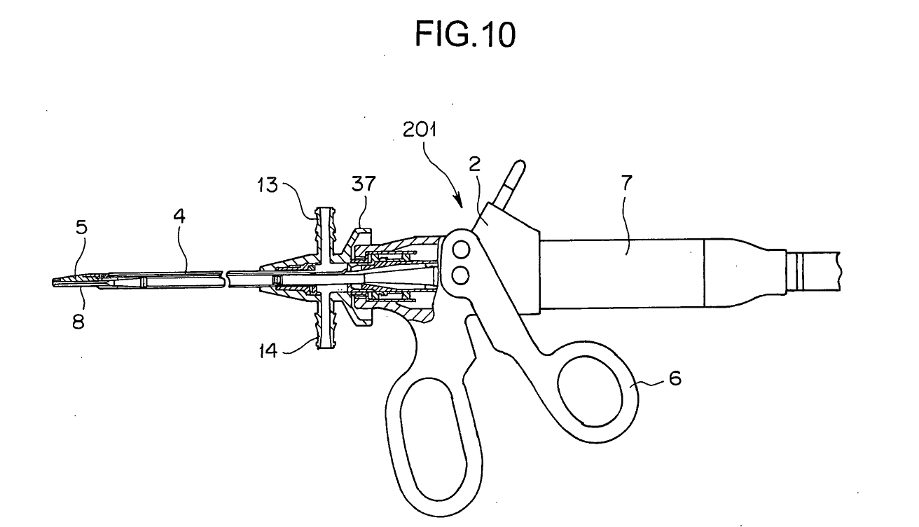

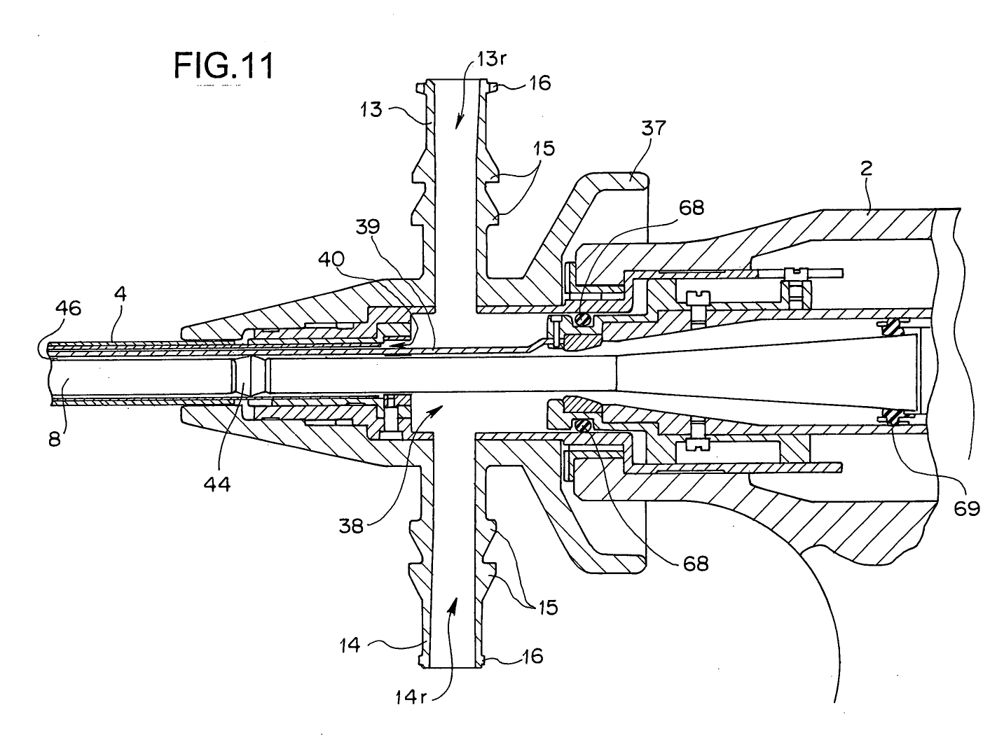

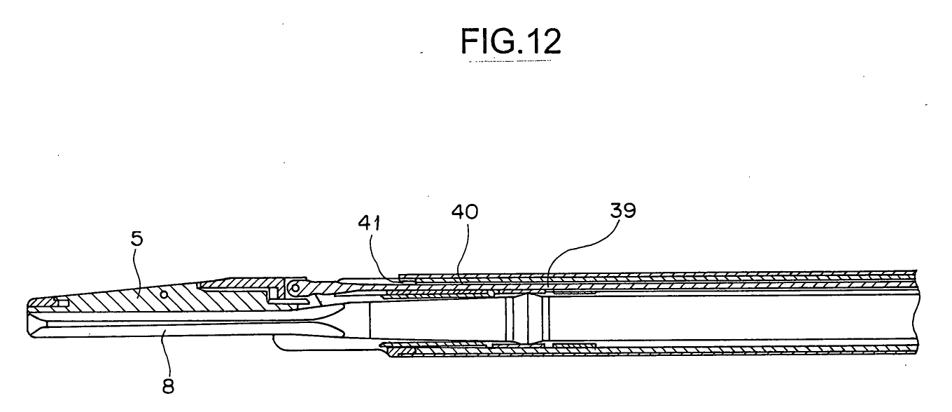

FIG. 10 is a view of an ultrasonic treatment instrument with only a part of an insertion portion in section showing a second embodiment of the present invention, FIG. 11 is an enlarged sectional view of the base end side of the insertion portion in FIG. 10, and FIG. 12 is an enlarged sectional view of the tip end side of the insertion portion in FIG. 10.

The construction of the ultrasonic treatment instrument of this embodiment is different from the ultrasonic treatment instrument 1 of the first embodiment shown in FIGS. 1 to 9 in the point that the water/air supply port 13 and the suction port 14 are not provided to the body portion 10 of the sheath 9 but provided on a rotating knob. Therefore, only the difference will be described and the same reference numerals are given to the same construction in the first embodiment, whose description will be omitted.

As shown in FIG. 10, an ultrasonic treatment instrument 201 comprises the elongated insertion portion 4 to be inserted into a body cavity, and the grip portion 5, which is a treatment portion, is disposed at the tip end side of the insertion portion 4. And, at least a part of the insertion portion 4, the base end side, for example, is covered by a rotating knob 37, which is a cylindrical member of an operation portion. As a result, the tip end sides of the grip portion 5 and the probe 8 are projected from the rotating knob 37. It is to be noted that the rotating knob 37 is to rotate/operate the insertion portion 4 in the direction around the axis of the insertion portion 4, and its function is substantially the same as that of the rotating knob 3 in the first embodiment.

Moreover, a space 38 as a flow passage is formed between the outer circumference on the base end side of the insertion portion 4 and the inner circumference of the rotating knob 37. That is, the base end side of the insertion portion 4 is inserted into the space 38 in the inner circumference of the rotating knob 37. Also, the rotating knob 37 is connected to the tip end of the fixed handle 2 of the operation portion.

Furthermore, the movable handle 6 for opening/closing operation of a pair of jaws of the grip portion 5 is rotatably mounted through a support shaft to the fixed handle 2. Also, the elongated probe 8 with the tip end connected to the grip portion 5 and the base end connected to the vibrator 7 and which is inserted into a pipeline probe channel 46 in the insertion portion 4 is mounted to the fixed handle 2.

Also, as shown in FIG. 11, a drive shaft channel 40 for transmitting the manipulation of the movable handle 6, for example, and through which a drive shaft 39 connecting the movable handle 6 to the grip portion 5 is inserted is disposed inside the insertion portion 4 between the inner circumference of the insertion portion 4 and the outer circumference of the pipeline probe channel 46. The drive shaft channel 40 also constitutes a flow passage.

The tip end side of the drive shaft channel 40 is, as shown in FIG. 12, opened as an opening portion 41 on the tip end side of the insertion portion 4 at a position opposite to the grip portion 5, for example. Also, the drive shaft channel 40 is inserted to the space 38 of the rotating knob 37. The opening portion 41 is provided at a position in the vicinity of the tip end side of the probe 8 from the grip portion 5. That is, the opening portion 41 is opened with respect at least to the tip end side of the probe 8.

Moreover, the water/air supply port 13 is provided to the rotating knob 37. The water/air supply port 13 has the flow passage 13r, and the flow passage 13r communicates to the space 38 of the rotating knob 37. That is, the flow passage 13r also communicates to the drive shaft channel 40. The water/air supply port 13 is connected to the water/air supply unit 29 through the tube 32 as in the above-mentioned first embodiment.

Moreover, the suction port 14 is provided to the rotating knob 37. The suction port 14 has the flow passage 14r, and the flow passage 14r communicates to the space 38 of the rotating knob 37. That is, the flow passage 14r also communicates to the drive shaft channel 40. The suction port 14 is connected to the suction unit 30 through the tube 32 as in the above-mentioned first embodiment.

Moreover, at a middle position of the probe 8 in the insertion portion 4, a rubber-lining 44 with an outer circumferential surface in close contact with the inner circumference of the pipeline probe channel 46 is provided so that the rubber-lining 44 covers the probe 8. Furthermore, a tip end ring 68 and a rubber-lining 69 are also disposed between a member disposed on the inner circumference of the fixed handle 2 and the probe 8 inserted into the fixed handle 2.

Since the construction of the ultrasonic treatment apparatus in which the so-constituted ultrasonic treatment instrument 201 is disposed is the same as that of the ultrasonic treatment apparatus in the first embodiment shown in FIG. 6, the explanation will be omitted.

Next, the operation of the so-constituted ultrasonic treatment instrument 201 of this embodiment will be described. Since the control operation of the ultrasonic treatment instrument 100 at a treatment is the same as that of the first embodiment shown in FIG. 8, the explanation will be omitted. Therefore, only a flow of a fluid in a flow passage of the ultrasonic treatment instrument 201 when supplying the fluid of water or air or sucking the fluid will be described as an operation of this embodiment.

First, the water/air supply port 13 and the water/air supply unit 29 are connected to each other by the tube 32 and moreover, the suction port 14 and the suction unit 30 are connected to each other by the tube 32, and then, the insertion portion 4 of the ultrasonic treatment instrument 201 is inserted into a body cavity to treat a tissue to be treated in the body cavity.

After that, when sucking the fluid such as mist generated in the treatment, on one hand, the fluid sucked by the suction unit 30 from the opening portion 41 advances into the drive shaft channel 40 and then, flows through the flow passages 38, 14r and is discharged by the suction unit 30 through the tube 32 connected to the suction port 14.

At this time, it is so constituted that the fluid having entered the rotating knob 37 does not flow into the pipeline probe channel 46 due to the rubber lining 44 provided to the probe 8. Also, it is so constituted that the sucked fluid does not flow into the inner circumference of the fixed handle 2 since the tip end ring 68 and the rubber-lining 69 are provided.

On the other hand, the fluid supplied from the water/air supply unit 29 advances into the flow passage 13r of the water/air supply port 13 through the tube 32 and then, flows through the space 38, the drive shaft channel 40 and is supplied to the grip portion 5 and the probe 8 from the tip end opening portion 41.

In this way, in the ultrasonic treatment instrument 201 showing this embodiment, the water/air supply port 13 and the suction port 14 are provided to the rotating knob 37. Also, the drive shaft channel 40 into which the drive shaft 39 disposed in the insertion portion 4 is inserted is used as a flow passage for supplying a fluid of water or air and sucking a fluid. That is, the drive shaft channel 40 serves as a flow passage for supplying a fluid of water or air and as a flow passage for sucking the fluid from within the body cavity.

By this, since a flow passage can be formed through which the fluid can flow into the ultrasonic treatment instrument 201 without using the sheath 9, manufacturing costs can be reduced as compared with the above-mentioned first embodiment.

Moreover, when rotating the rotating knob 37, the water/air supply port 13 and the suction port 14 can be moved to a position not in contact with rotational movement of the rotating knob.

The other effects are the same as those of the ultrasonic treatment instrument of the above-mentioned first embodiment.

### Third Embodiment

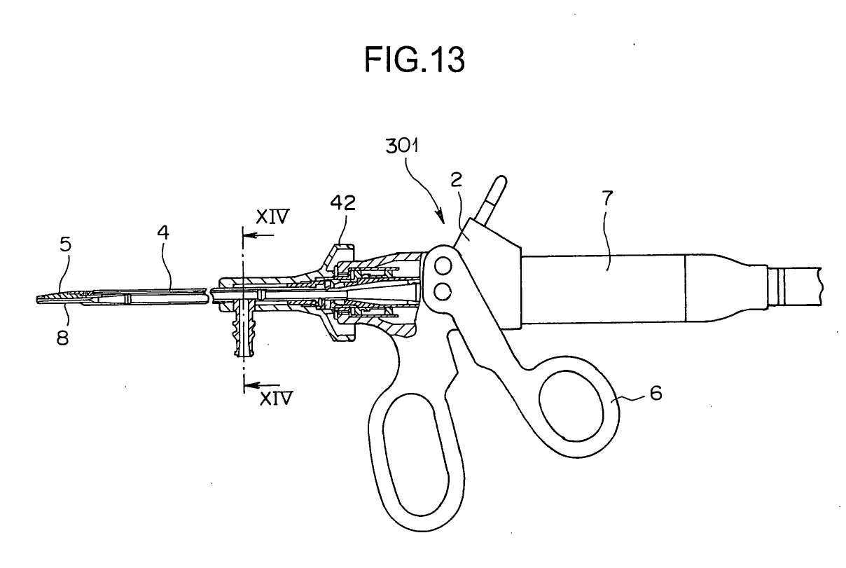

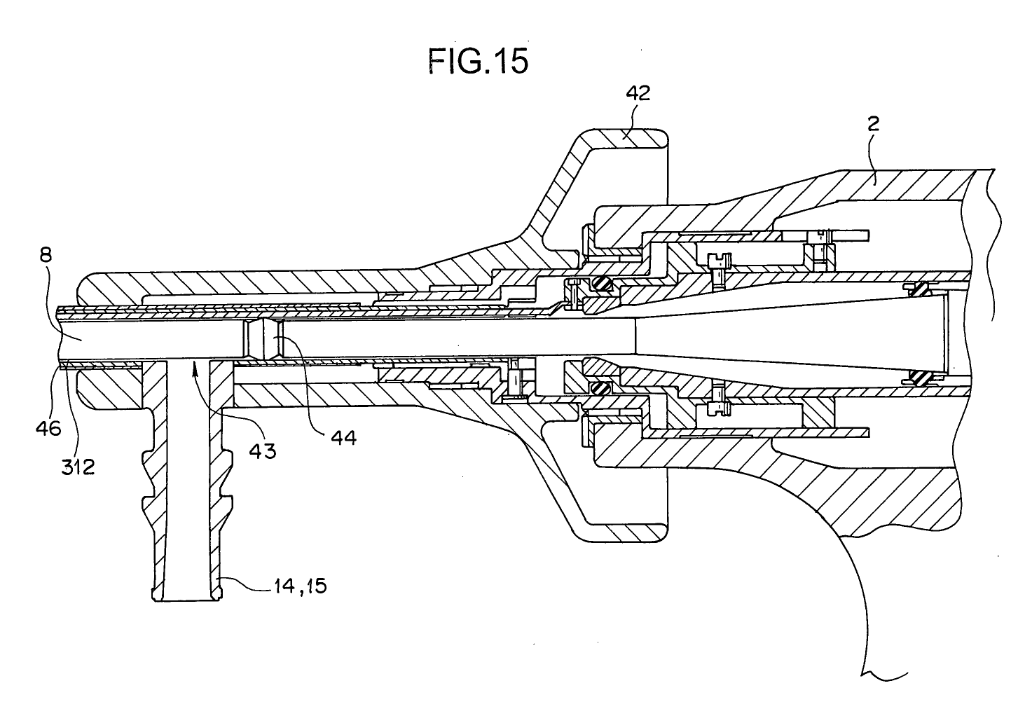

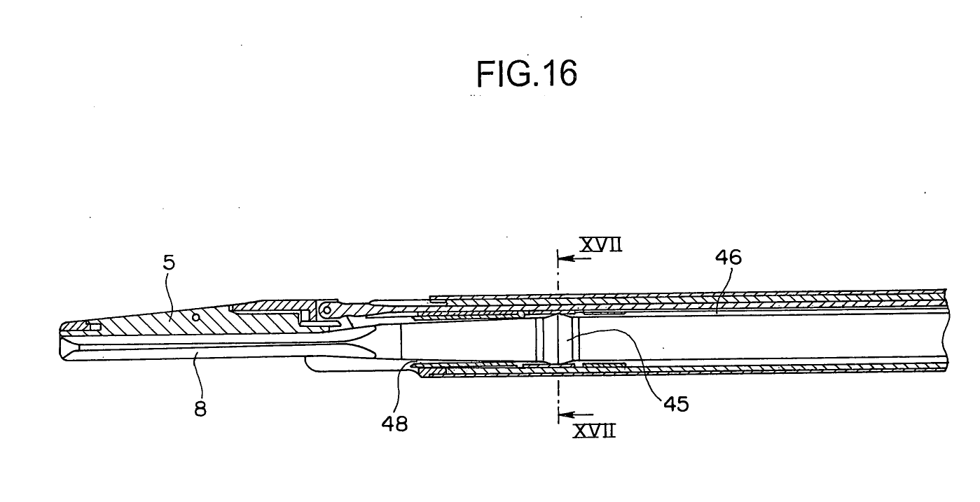

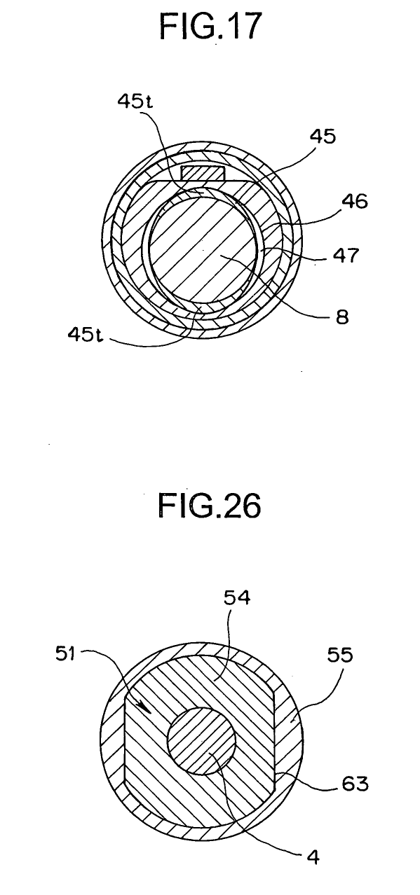

FIG. 13 is a view of an ultrasonic treatment instrument with only a part of an insertion portion in section showing a third embodiment of the present invention, FIG. 14 is a sectional view along XIV-XIV line in FIG. 13, FIG. 15 is an enlarged sectional view of the base end side of the insertion portion in FIG. 13, FIG. 16 is an enlarged sectional view of the tip end side of the insertion portion in FIG. 13, and FIG. 17 is a sectional view along XVII-XVII line in FIG. 16.

The construction of the ultrasonic treatment instrument of this embodiment is different from the ultrasonic treatment instrument 1 in the second embodiment shown in FIGS. 10 to 12 in the point that the rotating knob is extended toward the tip end side of the insertion axis and the water/air supply port 13 and the suction port 14 are provided at a portion of this extension. Therefore, only the difference will be explained, and the same reference numerals are given to the same construction in the second embodiment, whose description will be omitted.

As shown in FIG. 13, an ultrasonic treatment instrument 301 comprises the elongated insertion portion 4 to be inserted into a body cavity, and the grip portion 5, which is a treatment portion, is disposed at the tip end side of the insertion portion 4. Moreover, at least a part of the insertion portion 4, the base end side, for example, is covered by a rotating knob 42, which is a cylindrical member of an operation portion. That is, the tip end sides of the grip portion 5 and the probe 8 are projected from the rotating knob 42.

The rotating knob 42 is to rotate/operate the insertion portion 4 in the direction around the axis of the insertion portion 4, and its function is substantially the same as that of the rotating knob 37 of the second embodiment, but it is formed extending in the tip end direction of the insertion portion 4 as compared with the rotating knob 37 of the second embodiment. Moreover, the rotating knob 42 is connected to the tip end of the fixed handle 2 of the operation portion.

Furthermore, the movable handle 6 for opening/closing operation of a pair of jaws of the grip portion 5 is rotatably mounted on the fixed handle 2 through a support shaft. At the fixed handle 2, the elongated probe 8 which is inserted into the pipeline probe channel 46 in the insertion portion 4 and has its tip end connected to the grip portion 5 and the base end to the vibrator 7 is mounted. A flow passage 312 is formed between the inner circumference of the pipeline probe channel 46 and the outer circumference of the probe 8 as shown in FIG. 15.

Moreover, at a position on the base end side of the probe 8 in the insertion portion 4, the rubber-lining 44, which has an outer circumferential face with a sectional shape capable of close contact with the inner circumference of the pipeline probe channel 46, a circular section, for example, is provided.

Furthermore, at a position on the tip end side of the probe 8 in the insertion portion 4, a rubber-lining 45, which has an outer circumferential face with a sectional shape capable of close contact with the inner circumference of the pipe line probe channel 46, an oval section, for example, is provided.

In more detail, as shown in FIG. 17, the rubber-lining 45 with two contact portions 45t in contact with the inner circumference of the pipeline probe channel 46 covers the outer circumference of the probe 8 in the pipeline probe channel 46.

A tip end flow passage 47 is formed between the rubber-lining 45 which is divided by the two contact portions 45t and the inner circumference of the pipeline probe channel 46. The tip end flow passage 47 communicates to the flow passage 312 and is opened as an opening portion 48 at a position opposite to the grip portion 5, for example, at the tip end side of the insertion portion 4 as shown in FIG. 16. The opening portion 48 is provided at a position in the vicinity of the tip end side of the probe 8 from the grip portion 5. That is, the opening portion 48 is opened with respect at least to the tip end side of the probe 8.

It is to be noted that the rubber-lining 45 is not limited to an oval shape as long as the tip end flow passage 47 is formed between the rubber-lining 45 and the inner circumference of the pipeline probe channel 46.

At the portion of the rotating knob 42 extended toward the tip end side of the insertion axis, the water/air supply port 13 is provided in the state of being fitted in a hole 43 provided to the insertion portion 4 as shown in FIG. 14. At this time, the water/air supply port 13 is provided substantially perpendicular to the insertion portion 4 at the portion of the rotating knob 42 extended toward the tip end side of the insertion axis so that it does not interfere with the drive shaft channel 40.

The water/air supply port 13 has the flow passage 13r, and the flow passage 13r communicates to the flow passage 312. That is, the flow passage 13r also communicates to the tip end flow passage 47. The water/air supply port 13 is connected to the water/air supply unit 29 through the tube 32 as with the above-mentioned first and the second embodiments.

Also, at the portion of the rotating knob 42 extended toward the tip end side of the insertion axis, the suction port 14 is provided in the state of being fitted in the hole 43 provided to the insertion portion 4 as shown in FIG. 14. The suction port 14 is provided substantially perpendicular to the insertion portion 4 at the portion of the rotating knob 42 extended toward the tip end side of the insertion axis so that it does not interfere with the drive shaft channel 40.

The suction port 14 has the flow passage 14r, and the flow passage 14r communicates to the flow passage 312. That is, the flow passage 14r also communicates to the tip end flow passage 47. The suction port 14 is connected to the suction unit 30 by the tube 32 as with the above-mentioned first and the second embodiments.

Since the construction of the ultrasonic treatment apparatus in which the so-constituted ultrasonic treatment instrument 301 is disposed is the same as that of the ultrasonic treatment apparatus of the first embodiment shown in FIG. 6, the explanation will be omitted.

Next, the operation of the so-constituted ultrasonic treatment instrument 301 of this embodiment will be described. Since the control operation of the ultrasonic treatment apparatus 100 at a treatment is the same as the control operation of the first embodiment shown in FIG. 8, the explanation will be omitted. Therefore, only a flow of a fluid in a flow passage of the ultrasonic treatment instrument 301 at suction of a fluid and at supply of a fluid of water or air will be described as the operation of this embodiment.

First, the water/air supply port 13 and the water/air supply unit 29 are connected to each other by the tube 32, and moreover, the suction port 14 and the suction unit 30 are connected to each other by the tube 32 and then, the insertion portion 4 of the ultrasonic treatment instrument 301 is inserted into a body cavity to treat a tissue to be treated in the body cavity.

After that, when sucking a fluid such as mist generated in a treatment, on one hand, the fluid sucked by the suction unit 30 from the opening portion 48 advances into the tip end flow passage 47 and then, flows through the flow passages 312, 14r and is discharged by the suction unit 30 through the tube 32 connected to the suction port 14.

At this time, it is so constituted that the fluid having entered the pipeline probe channel 46 does not flow into the fixed handle 2 due to the rubber-lining 44.

On the other hand, the fluid supplied from the water/air supply unit 29 advances into the flow passage 13r of the water/air supply port 13 through the tube 32 and then, flows through the flow passage 312 and the tip end flow passage 47 and is supplied to the grip portion 5 or the probe 8 from the opening portion 48.

By constituting the ultrasonic treatment instrument 301 of this embodiment in this way, the same effect as that of the ultrasonic treatment instrument 201 of the above-mentioned second embodiment can be obtained.

A variation will be described below.

FIG. 18 is a partial sectional view showing a variation in which a groove is provided at a position opposite to the grip portion on the probe of ultrasonic treatment instruments in FIGS. 1, 10 and 13.

As shown in FIG. 18, five grooves 49, for example, may be formed at a tip end portion of the probe 8 to be connected to the grip portion 5 and on a back surface 8r of a connection surface. By forming the five grooves 49 on the back surface 8r of the probe 8 to be connected to the grip portion 5 in this way, a surface area of the back surface 8r of the probe 8 is increased by the grooves 49, and heat accumulated in the probe 8 is easily discharged after a treatment, and in addition of supply of a fluid of water or air to the probe 8, the temperature of the probe 8 can be lowered.

Also in this case, a fluid of water or air may be supplied to the probe 8. Moreover, the number of the grooves 49 is not limited to five, but the grooves 49 may be formed in any number as long as it is not on a surface to be meshed with the grip portion 5. The shape or direction of the groove 49 may be set arbitrarily.

Another variation will be described below.

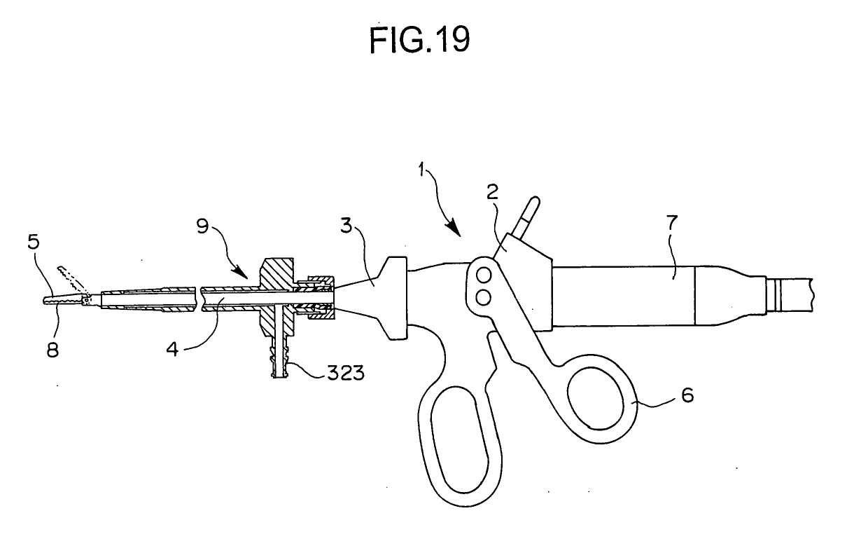

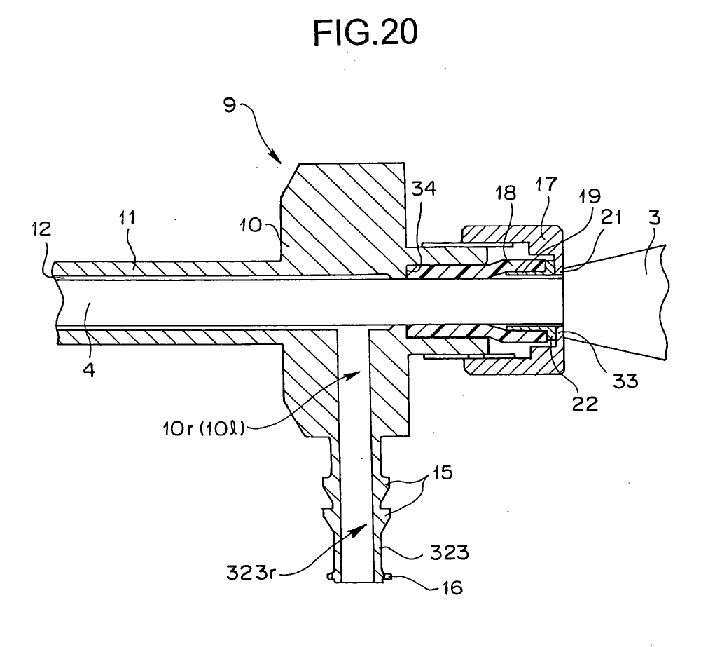

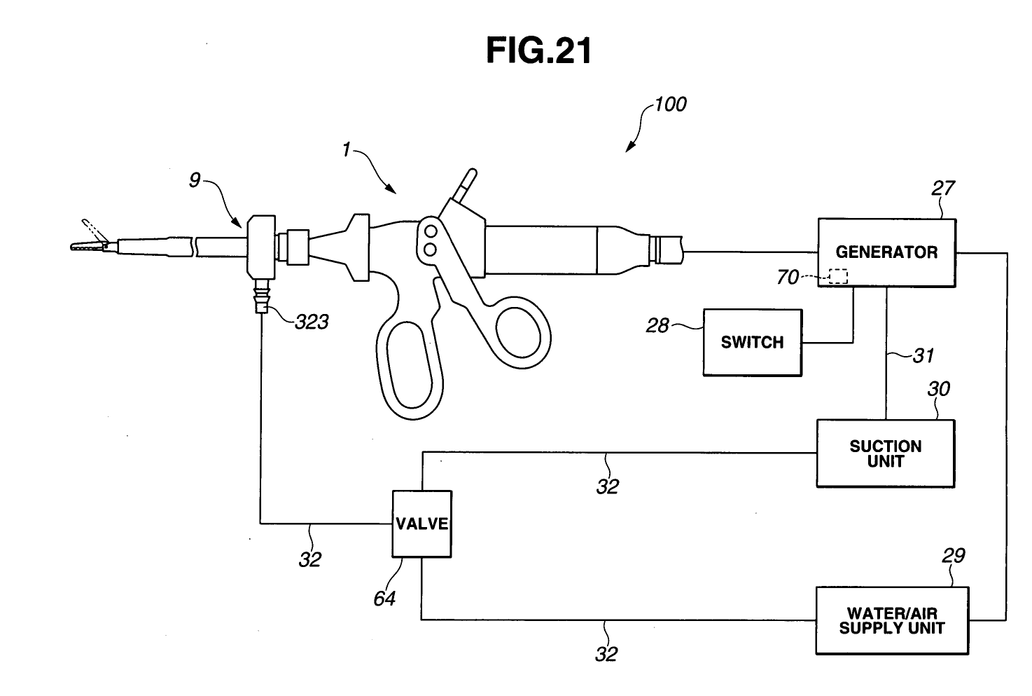

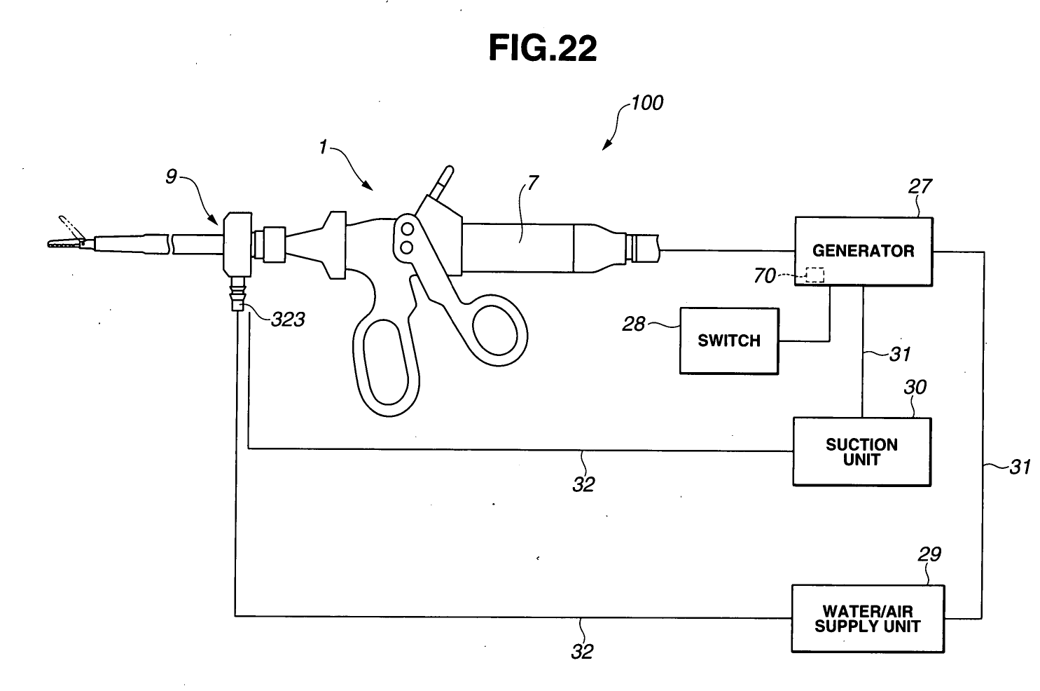

FIG. 19 is a partial sectional view showing a variation in which a single port into which a water/air supply port and a suction port are made common is provided to the ultrasonic treatment instrument 1 in FIG. 1, FIG. 20 is an enlarged sectional view of the base end side of the insertion portion in FIG. 19, FIG. 21 is a block diagram schematically showing a construction of an ultrasonic treatment apparatus having the ultrasonic treatment instrument in FIG. 19, and FIG. 22 is a block diagram schematically showing another construction of the ultrasonic treatment apparatus in FIG. 21.

In the ultrasonic treatment instrument 1 in the above-mentioned first embodiment, two ports of the water/air supply port 13 and the suction port 14 are provided to the body portion 10. Not limited to this, only a single port 323 into which the water/air supply port 13 and the suction port 14 are made common may be provided to the body portion 10. That is, the port 323 constitutes an inlet/outlet port.

Specifically, as shown in FIG. 19, the common port 323 for connection to the water/air supply unit 29 (See FIG. 21) and the suction unit 30 (See FIG. 21) is provided to the body portion 10. As shown in FIG. 20, the common port 323 has a flow passage 323r, and the flow passage 323r communicates to the flow passage 10r (101) of the body portion 10. That is, the flow passage 323r also communicates to the flow passage 12.

The common port 323 constitutes an inlet/outlet port for performing at least one of supply to the flow passage 12 of a fluid to be supplied to a treatment portion made of the tip end sides of the grip portion 5 and the probe 8 and discharge from the flow passage 12 of the fluid sucked from within the body cavity.

Moreover, as shown in FIG. 21, the common port 323 is connected to the water/air supply unit 29 and the suction unit 30 by the tube 32 provided with a switching valve 64 at a middle position. At this time, one end of the tube 32 covers an outer circumference of the common port 323. The switching valve 64 is to switch between an operation for supplying a fluid of water or air from the water/air supply unit 29 and an operation for sucking the fluid by the suction unit 30.

The connection between the common port 323 to the water/air supply unit 29 and the suction unit 30 by the tube 32 may be constituted so that, as shown in FIG. 22, the water/air supply unit 29 and the suction unit 30 are connected individually to the common port 323. In this case, the ultrasonic treatment apparatus 100 can be constituted with a simple mechanism and more inexpensively than using the switching valve 64.

Here, if a fluid of water or air is supplied from the water/air supply unit 29 on one hand, the fluid advances into the flow passage 323r of the common port 323 through the tube 32 connected to the water/air supply unit 29 by means of the switching valve 64 and then, passes through the flow passage 10r (101), 12 and is supplied from the tip end opening portion 35 (See FIG. 4) of the tip end flow passage 26 (See FIG. 5) to the grip portion 5 or the probe 8.

On the other hand, when the fluid is sucked by the suction unit 30 from the tip end opening portion 35, the fluid advances into the tip end flow passage 26 from the tip end opening portion 35 by means of the switching valve 64, passes through the flow passage 12, 10r (101) and is discharged from the suction unit 30 through the tube 32 connected to the common port 323.

Even by providing only a single port for supplying water or air and for sucking on the ultrasonic treatment instrument in this way, the same effect as that of the above-mentioned first embodiment can be obtained. Such a construction may be also applied to the ultrasonic treatment instrument 201 of the above-mentioned second embodiment or the ultrasonic treatment instrument 301 of the third embodiment.

### Fourth Embodiment

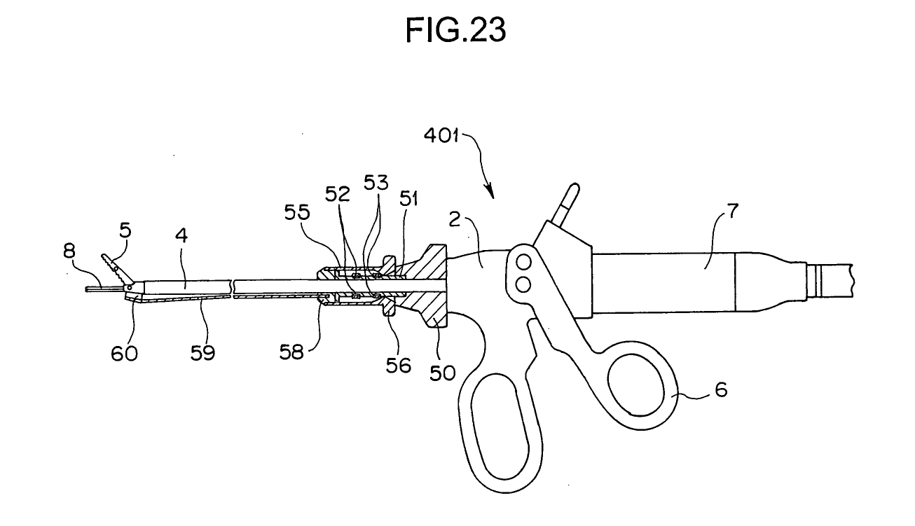

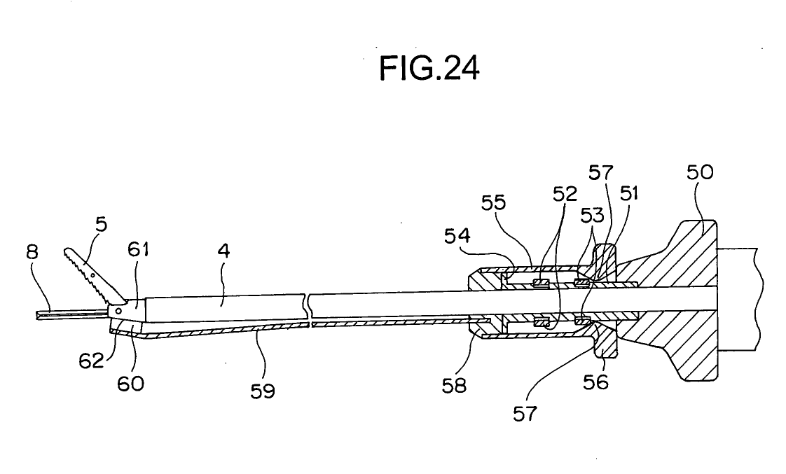

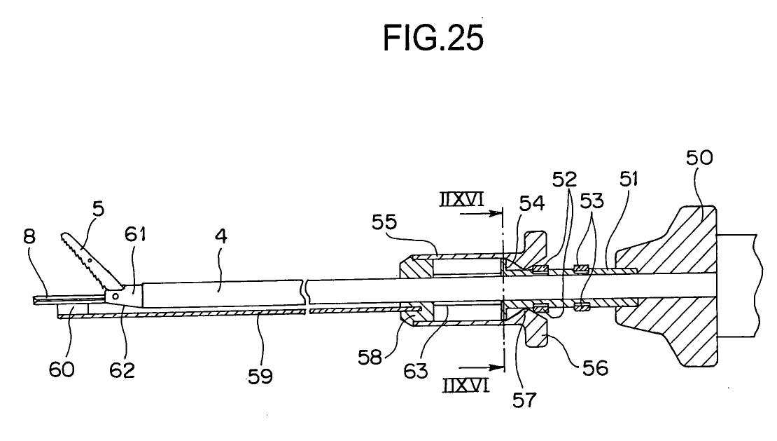

FIG. 23 is a view of an ultrasonic treatment instrument with only a part of an insertion portion in section showing a fourth embodiment of the present invention, FIG. 24 is an enlarged view of the tip end side of the insertion portion in FIG. 23, FIG. 25 is a view showing a state where a slider member in FIG. 24 is moved to the tip end in an axial direction of insertion, and FIG. 26 is a sectional view along IIXVI-IIXVI line in FIG. 25.

The construction of the ultrasonic treatment instrument of this embodiment is different from the ultrasonic treatment instrument 1 of the first embodiment shown in FIGS. 1 to 9, the ultrasonic treatment instrument 201 of the second embodiment shown in FIGS. 10 to 12 and the ultrasonic treatment instrument 301 of the third embodiment shown in FIGS. 13 to 17 in the point that a fluid is not used in cooling the grip portion 5 and the probe 8. Therefore, only the difference will be described, and the same reference numerals are given to the same construction in the first to the third embodiments, whose description will be omitted.

As shown in FIG. 23, an ultrasonic treatment instrument 401 comprises the elongated insertion portion 4 to be inserted into a body cavity, and the grip portion 5, which is a treatment portion, is disposed at the tip end side of the insertion portion 4. The base end side of the insertion portion 4 is covered by a rotating knob 50 of the operation portion.

The rotating knob 50 is to rotate/operate the insertion portion 4 in the direction around the axis of the insertion portion 4, and its function is substantially the same as that of the rotating knob 3 in the first embodiment. Moreover, the rotating knob 50 is connected to the tip end of the fixed handle 2 of the operation portion.

Furthermore, the movable handle 6 for opening/closing operation of a pair of jaws of the grip portion 5 is rotatably mounted to the fixed handle 2 through a support shaft. Also, the elongated probe 8 with the tip end connected to the grip portion 5 and the base end to the vibrator 7 is mounted to the fixed handle 2.

Moreover, a slider receiver 51 covering the insertion portion 4 is provided at the base end side of the insertion portion 4 and in the vicinity of the tip end of the rotating knob 50, and the slider receiver 51 has its base end portion fixed between an inner circumference on the tip end side of rotating knob 50 and the outer circumference of the insertion portion 4.

The tip end of the slider receiver 51 is covered by a stopper 54, and between the stopper 54 of the slider receiver 51 and the base end portion of the slide receiver 51 is covered by C rings 52, 53, respectively.

The outer circumference of the insertion portion 4 is covered by a slider member 55 so that it covers the slider receiver 51. At the base end portion of the slider member 55, a flange-state retaining portion 56 is provided, and on an inner circumference of the retaining portion 56, a projection portion 57 to be slidably brought into contact with the slider receiver 51 is formed as shown in FIG. 24.

Furthermore, as shown in FIGS. 25, 26, on a portion of the outer circumference of the insertion portion 4 covered by the slider member 55 and in which the slider receiver 51 is not provided, a straight slider 63 is formed along the insertion portion 4.

Also, a connection portion 58 is formed at the tip end portion of the slider member 55, and a base end portion of an elongated plate spring 59 along the insertion portion 4 is connected to the connection portion 58. At the tip end portion of the plate spring 59, a cooling portion 60 such as a wetted gauze is provided, and the cooling portion 60 is normally brought into contact with a tapered surface 62 formed on a tip end cover 61 supporting the grip portion 5 as shown in FIG. 24.

Next, the operation of the so-constituted embodiment will be described.

After an ultrasonic treatment of a tissue to be treated in a body cavity by the grip portion 5 is finished, by sliding the slider member 55 from a position shown in FIG. 24 to a position shown in FIG. 25, the cooling portion 60 in contact with the tapered surface 62 is brought into contact with the tip end side of the probe 8. By this, the tip end side of the probe 8 is cooled.

At this time, the slider member 55 is fixed since movement in the insertion axial direction of the projection portion 57 is regulated by the stopper 54 and the C ring 52, as shown in FIG. 25.

After cooling the probe 8, the slider member 55 is slid from the position shown in FIG. 25 to the position shown in FIG. 24. At this time, by moving the base end side of the retaining portion 56 of the slider member 55 till it abuts against the tip end side of the rotating knob 50, the projection portion 57 is slid to the base end side while crushing the C rings 52, 53.

Moreover, when the slider member 55 is slid, the connection portion 58 and the plate spring 59 are also moved to the base end side at the same time. When the connection portion 58 is slid, it is guided by the slider 63 provided on the outer circumference of the insertion portion 4 so that it slides along the insertion axis without rotation.

Moreover, the cooling portion 60 is slid toward the base end side while sliding on the tapered surface 62 of the tip end cover 61. At this time, the tip end side of the plate spring 59 is curved as shown in FIG. 24.

The slider member 55 having been moved to the position in FIG. 24 is fixed since the movement in the insertion axial direction of the projection portion 57 is regulated by the C ring 53 and the rotating knob 50.

In this way, in the ultrasonic treatment instrument 401 of this embodiment, when cooling the probe 8 after a treatment, it is shown that the cooling portion 60 is brought into contact with the probe 8 by sliding the slider member 55 covering the insertion portion 4 in the insertion axial direction without using a fluid.

By this, since the probe whose temperature has become high due to frictional heat by an ultrasonic treatment can be cooled without connecting the ultrasonic treatment instrument with the water/air supply unit or the suction unit, the probe after the treatment can be cooled with a reduced cost.

In this case, smoke or mist generated during the ultrasonic treatment can not be sucked, but by combining this embodiment with the above-mentioned first to the third embodiments in use, the smoke or mist can be also sucked.

A variation will be shown below.

In the above-mentioned first to the fourth embodiments, an ultrasonic treatment instrument for coagulation ablation was used as an example of a medical treatment instrument, but it is needless to say that the application is not limited to this but this embodiment can be applied to any medical treatment instruments using heat or endoscopic medical treatment instruments.

Specifically, it is needless to say that this embodiment can be applied to high-frequency treatment instruments using Joule heat or the like by high-frequency electric current, laser treatment instrument using laser beam, RF treatment instruments using electromagnetic wave, or treatment instruments used by generating heat by heating forceps.

### Fifth Embodiment

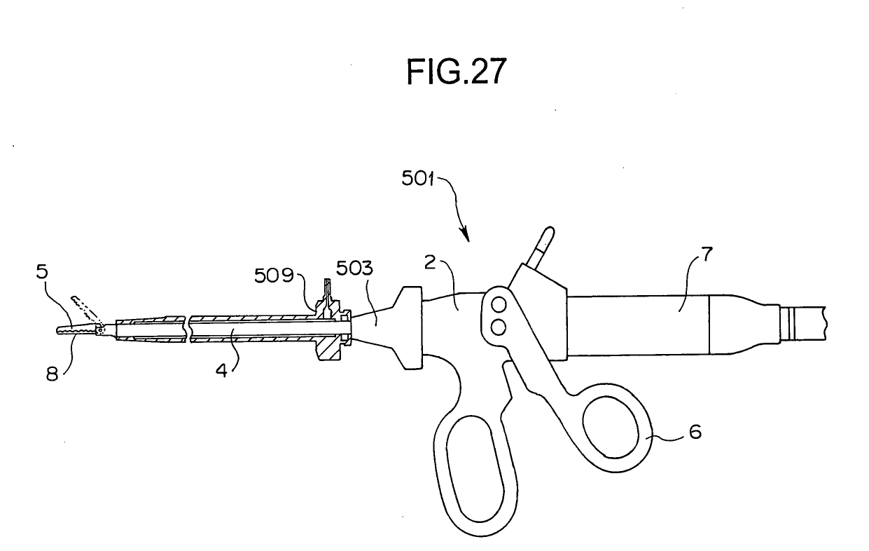

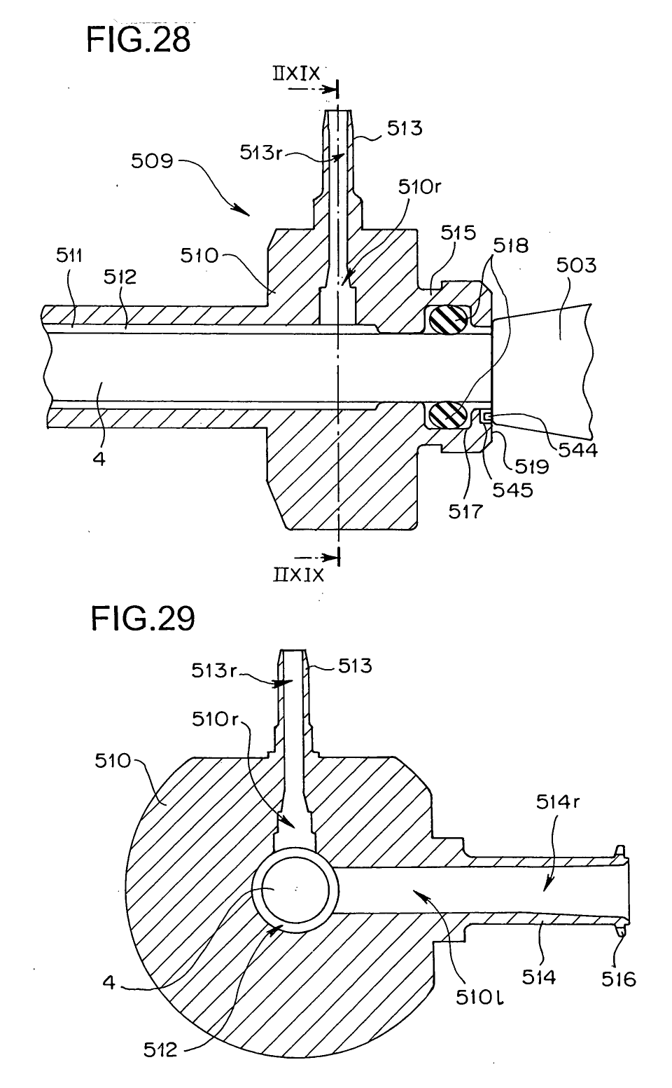

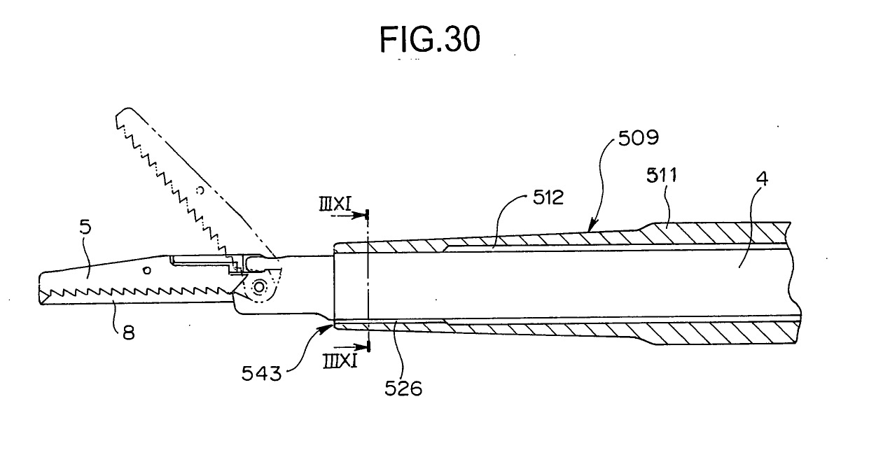

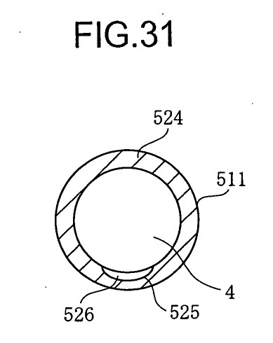

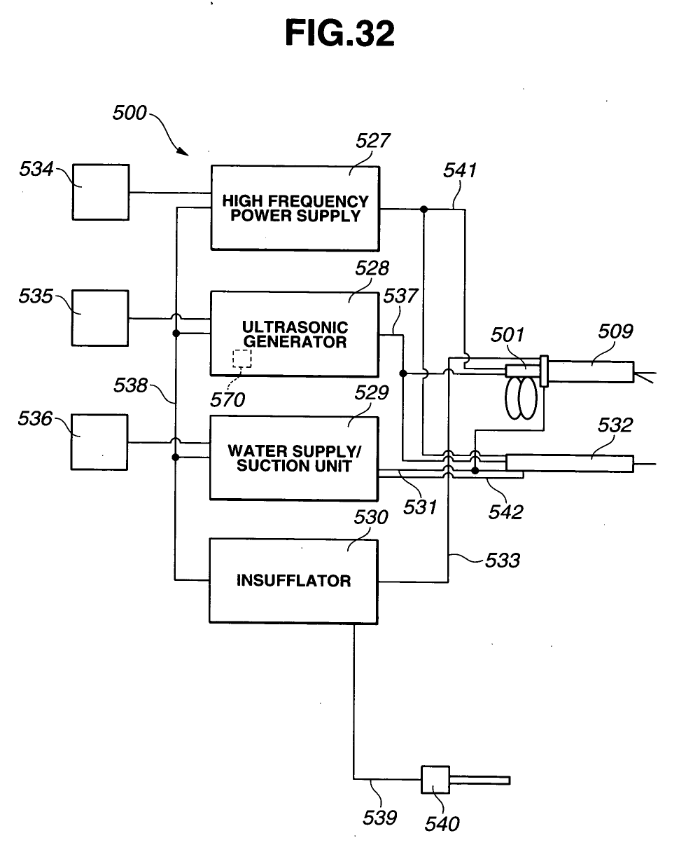

FIG. 27 is a view of an ultrasonic treatment instrument with only a part of an insertion portion in section showing a fifth embodiment of the present invention, FIG. 28 is an enlarged sectional view of the base end side of the insertion portion in FIG. 27, FIG. 29 is a sectional view along IIXIX-IIXIX line in FIG. 28, FIG. 30 is a enlarged sectional view of the tip end side of the insertion portion in FIG. 27, FIG. 31 is a sectional view along IIIXI-IIIXI line in FIG. 30, and FIG. 32 is a block diagram schematically showing a construction of an ultrasonic treatment apparatus having the ultrasonic treatment instrument in FIG. 27.

As shown in FIG. 27, an ultrasonic treatment instrument 501 comprises the elongated insertion portion 4 to be inserted into a body cavity, and the grip portion 5, which is a treatment portion, is disposed at the tip end side of the insertion portion 4. The grip portion 5 comprises a pair of jaws capable of being opened/closed, for example, and the jaws grip and heat a tissue to be treated in the body cavity and perform a treatment such as coagulation ablation.

Moreover, the base end side of the insertion portion 4 is connected to a tip end of a rotating knob 503, which is an operation portion. The rotating knob 503 is connected to the tip end of the fixed handle 2, which is the operation portion. The rotating knob 503 is to rotate and operate the insertion portion 4 in the direction around the axis of the insertion portion 4.

Furthermore, the movable handle 6 for opening/closing operation of the pair of jaws of the grip portion 5 is rotatably mounted to the fixed handle 2 through a support shaft. Also, the elongated probe 8 with the tip end connected to the grip portion 5 and the base end to the vibrator 7 is inserted into the insertion portion 4 and mounted to the fixed handle 2.

The probe 8 is to perform various treatments such as coagulation for a tissue to be treated using a heat generated by electric power, which is an electric energy supplied from the generator, and a portion at the tip end side of the probe 8 connected to the grip portion 5 constitutes a treatment portion.

Moreover, a water supply/suction sheath 509, which is a cylindrical member, is attached on the outer circumference of the insertion portion 4, specifically the outer circumference excluding the tip end sides of the grip portion 5 of the insertion portion 4 and the probe 8. The tip end sides of the grip portion 5 and the probe 8 are projected from the sheath 509. The sheath 509 is detachably attached to at least a part of the outer circumference of the insertion portion 4. That is, at least a part of the insertion portion 4 is inserted into the sheath 509.

As shown in FIG. 28, the sheath 509 has a major part comprised by a flange-state body portion 510, for example, an insertion portion 511 extended to the tip end side from substantially the center of the body portion 510 and having a diameter smaller than that of the body portion 510, and a connection portion 515 extended to the base end side from substantially the center of the body portion 510 and having a diameter smaller than that of the body portion 510.

An inner diameter of a hole of the insertion portion 511 is formed larger than the outer diameter of the insertion portion 4. By this, a flow passage 512 is formed between an inner circumference of the insertion portion 511 and the outer circumference of the insertion portion 4. Moreover, flow passages 510r, 5101 communicating from the tip end to the base end through the body portion 510 and substantially perpendicular to the hole are formed. The flow passages 510r, 5101 communicate to the flow passage 512.

Furthermore, a water supply port 513, which is an inlet to be connected to a water supply/suction unit 529 (See FIG. 32), which will be described later, is provided to the body portion 510 so that it is substantially perpendicular to the insertion portion 511. The water supply port 513 has a flow passage 513r, and the flow passage 513r communicates to the flow passage 510r of the body portion 510. That is, the flow passage 513r also communicates to the flow passage 512. The water supply port 513 may be connectable to an air supply unit, not shown.

Moreover, as shown in FIG. 29, a suction port 514, which is an outlet to be connected to an insufflator 530 (See FIG. 32) having a scavenging function, which will be described later, is provided to the body portion 510 so that it is substantially perpendicular to the insertion portion 511. The suction port 514 has a flow passage 514r, and the flow passage 514r communicates to the flow passage 5101 of the body portion 510. That is, the flow passage 514r also communicates to the flow passage 512.

The water supply port 513 and the suction port 514 may be provided not limited to the state substantially perpendicular to the insertion portion 511 but with a free mounting angle to the body portion 510 as long as it is in a range not interfering with the ultrasonic treatment instrument 501.

Moreover, the water supply port 513 and the suction port 514 constitute an inlet/outlet for performing at least one of supply to the flow passage 512 of a fluid to be supplied to the treatment portion comprised by the tip end sides of the grip portion 5 and the probe 8 and discharge from the flow passage 512 of the fluid sucked from within the body cavity.

An outer circumference of the water supply port 513 is covered by one end of a water supply tube 531 (See FIG. 32), which will be described later, for connecting the water supply port 513 and the water supply/suction unit 529. The water supply tube 531 may be connectable not only to the water supply/suction unit 529 but also to an ultrasonic suction treatment instrument 532 shown in FIG. 32. In this case, the water supply port 513 is formed in the common shape with a water supply port, not shown, of the ultrasonic suction treatment instrument 532.

As shown in FIG. 29, to the projected tip end of the suction port 514, a luer-lock base 516 is provided to be connected to a luer-lock connector, not shown, of an exhaust tube 533, which will be described later, so that the exhaust tube 533 for connecting the suction port 514 and the insufflator 530 can be connected thereto.

As shown in FIG. 28, a recess portion 517 opened toward the base end side is provided to the connection portion 515 of the body portion 510, and an elastic member 518 made of an O-ring or the like, for example, is fitted in the recess portion 517.

An inner diameter of a hole of the O-ring of the elastic member 518 is formed smaller than the outer diameter of the insertion portion 4 to such an extent that the insertion portion 4 can be inserted. When the insertion portion 4 is inserted into the hole of the O-ring of the elastic member 518, the elastic member 518 is elastically deformed between an inner circumferential face of the recess portion 517 and the insertion portion 4, and air tightness between the insertion portion 4 and the elastic member 518 can be maintained. That is, the elastic member 518 is brought into close contact with the outer circumference of the insertion portion 4 in the elastically deformed state.

Moreover, the opening of the recess portion 517 is formed at a base end portion 519 of the connection portion 515, and an inner circumference of the opening is formed larger than the outer diameter of the insertion portion 4 and smaller than the diameter on the tip end side of the rotating knob 503.

At the tip end of the rotating knob 503 of the ultrasonic treatment instrument 501, an engaging member 544, which is at least one engaging means, is provided, and at the base end portion 519 of the connection portion 515 of the sheath 509, an engagement receiving portion 545, which is engaging means to be engaged with the engaging member 544, is provided.

The engaging member 544 and the engagement receiving portion 545 are provided at the base end portion 519 and the rotating knob 503, respectively, so that when the engaging member 544 and the engagement receiving portion 545 are engaged with each other after the insertion portion 4 of the ultrasonic treatment instrument 501 is inserted into the sheath 509, as shown in FIG. 30, a tip end opening portion 543 of the sheath 509 is located on the probe 8 side at a lower side in FIG. 30.

When the insertion portion 4 is inserted into the sheath 509 and the rotating knob 503 is rotated in the state where the engaging member 544 is engaged with the engagement receiving portion 545, the engagement between the engaging member 544 and the engagement receiving portion 545 rotates the rotating knob 503 and the sheath 509 in the interlocking manner.

At this time, since positional relationship between the tip end opening portion 543 of the sheath 509 and the probe 8 is not changed since the rotating knob 503 and the sheath 509 are rotated in the interlocking manner, that is, the opening portion 543 is located in the vicinity of the probe 8 and the grip portion 5 all the time, the fluid having passed the flow passages 513r, 510r, 512, 526 is surely supplied to the probe 8 and the grip portion 5, and as a result, the probe 8 and the grip portion 5 are cooled. The opening portion 543 is located at a position in the vicinity on the tip end side of the probe 8 from the grip portion 5. That is, the opening portion 543 is opened at least toward the tip end side of the probe 8.

As shown in FIG. 30, the tip end of the sheath 509 when the sheath 509 is attached to the insertion portion 4 is located nearer to the base end side than the grip portion 5 so that the tip end sides of the grip portion 5 and the probe 8 are exposed. Moreover, the tip end side of the sheath 509 is formed in the tapered state.

As shown in FIG. 31, a support portion 524 in contact with the outer circumference of the insertion portion 4 is formed on an inner circumference on the tip end side of the insertion portion 511, and an arc 525 is also formed on the inner circumference on the tip end side of the insertion portion 511. The arc 525 is also located at a lower side in FIG. 30, that is on the probe 8 side when the sheath 509 is attached to the insertion portion 4 due to the engagement position between the engaging member 544 and the engagement receiving portion 545.

A tip end flow passage 526 is formed between the arc 525 and the outer circumference of the insertion portion 4. The tip end flow passage 526 communicates to the flow passage 512 formed between the inner circumference of the insertion portion 511 and the outer circumference of the insertion portion 4.

As shown in FIG. 32, an ultrasonic treatment apparatus 500 has a major part comprised by the ultrasonic treatment instrument 501, the ultrasonic suction treatment instrument 532, a high frequency power supply 527, an ultrasonic generator 528, the water supply/suction unit 529, the insufflator 530, a high frequency switch 534, an ultrasonic vibration switch 535 and a water supply/suction switch 536.

The vibrator 7 of the ultrasonic treatment instrument 501 is connected to the ultrasonic generator 528 for performing ultrasonic driving by an ultrasonic cable 537. Moreover, a vibrator, not shown, of the ultrasonic suction treatment instrument 532 can be also connected to the ultrasonic generator 528. Also, the ultrasonic vibration switch 535 for oscillating the vibrator 7 is connected to the ultrasonic generator 528.

The ultrasonic generator 528, the high frequency power supply 527, the water supply/suction unit 529 and the insufflator 530 are electrically connected to each other by a communication cable 538 so that they can be operated in the interlocking manner. Moreover, the water supply/suction unit 529 is connected to the water supply port 513 of the ultrasonic treatment instrument 501 by the water supply tube 531. The water supply tube 531 connected to the water supply/suction unit 529 can be connected to a water supply port, not shown, of the ultrasonic suction treatment instrument 532.

The insufflator 530, which is a suction portion, is connected to the suction port 514 of the ultrasonic treatment instrument 501 by the exhaust tube 533. The insufflator 530 is further connected to a known trocar 540 by an air supply tube 539. The exhaust tube 533 can be also connected to the trocar 540.

Moreover, the high frequency power supply 527 can be connected to the ultrasonic treatment instrument 501 and the ultrasonic suction treatment instrument 532, respectively, by a high frequency cable 541. Furthermore, the water supply/suction unit 529 can be connected to the ultrasonic suction treatment instrument 532 by a suction tube 542.

When a fluid is supplied by the water supply/suction unit 529, the fluid advances into the flow passage 513r of the water supply port 513 through the water supply tube 531 connected to the water supply/suction unit 529, passes the flow passage 510r, the flow passage 512 and then, supplied to the tip end sides of the grip portion 5 and the probe 8 from the tip end opening portion 543 of the tip end flow passage 526. In this case, the water supply/suction unit 529 may be an air supply unit, and the carried fluid may be gas.

Here, an opening area of the tip end opening portion 543 is formed smaller than a pipeline area of the flow passage 512. Therefore, the tip end flow passage 526 is narrowed in the nozzle state at the tip end opening portion 543, and a flow velocity of the fluid supplied from the tip end opening portion 543 is accelerated.

That is because, when a sectional area of the flow passage 512 is A1 and a sectional area of the opening portion 543 is A2, according to the well-known Bernoulli's theory, the relationship of V1A1=V2A2 (V1: Flow velocity in the flow passage, V2: Flow velocity of the opening portion) is true. And from the relationship of the flow velocity V2=V1 (A1/A2) when V1 is constant, according to the well-known venturi effect, V2 becomes large if a value of the opening sectional area A2 is smaller than that of the pipeline sectional area A1.

Therefore, as with this embodiment, by providing the opening portion 543 with an area smaller than the pipeline area of the flow passage 512 at one location, the flow velocity V2 at the opening portion 543 becomes larger than the flow velocity V1 at the flow passage 512.

Particularly when the outer diameter of the sheath 509 of the ultrasonic treatment instrument 501 is designed within the range of 5 to 10 mm, it is preferable that the sectional area A2 is set to not more than 30% of the sectional area A1, more effectively to 5 to 10%.

Moreover, since the tip end opening portion 543 is, as mentioned above, arranged on the probe 8 side, the fluid is surely supplied both to the probe 8 and the grip portion 5.

On the other hand, when a fluid such as smoke, mist, etc. is sucked by the insufflator 530 from the tip end opening portion 543, the fluid advances into the tip end flow passage 526 from the tip end opening portion 543 and then, passes through the flow passages 512, 5101, 514r and is discharged from the insufflator 530 through the exhaust tube 533 connected to the suction port 514.

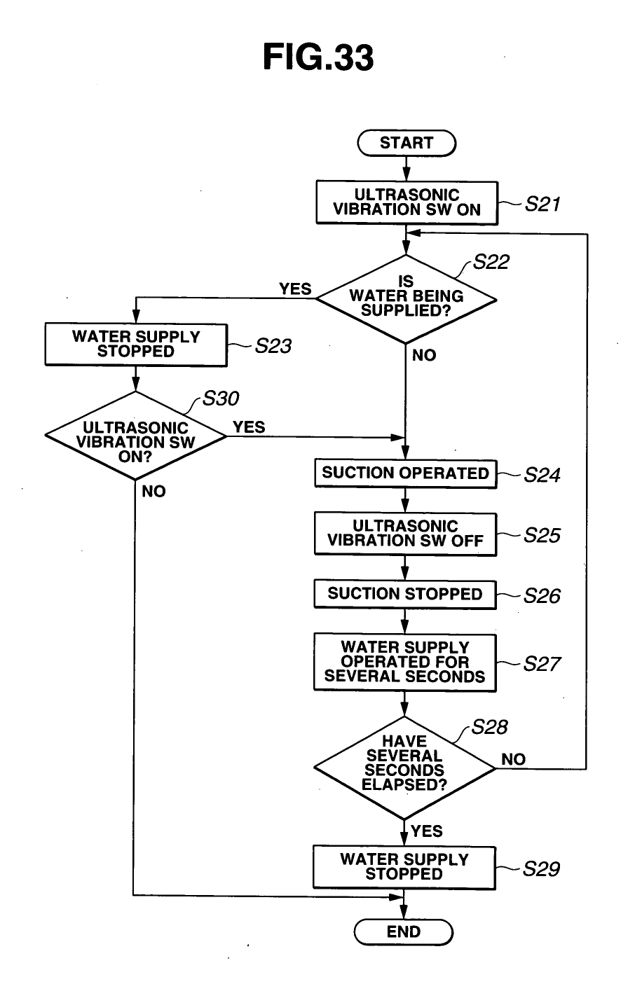

Next, the operation of the so-constituted ultrasonic treatment instrument 501 of this embodiment will be described using FIGS. 27 to 32 and FIG. 33. FIG. 33 is a flowchart showing a control method of the ultrasonic treatment apparatus when treating a tissue to be treated in a body cavity using the ultrasonic treatment instrument in FIG. 27.

First, to the insertion portion 4 of the ultrasonic treatment instrument 501, the sheath 509 is attached from the base end portion side of the sheath 509 toward the tip end side of the insertion portion 4 till it abuts against the rotating knob 503 and when the engaging member 544 is engaged with the engagement receiving portion 545, as shown in FIG. 28, the elastic member 518 is elastically deformed between the inner circumferential face of the recess portion 517 and the outer circumferential face of the insertion portion 4 and the elastic member 518 is brought into close contact with the insertion portion 4. At this time, as mentioned above, the flow passage 512 is formed between the outer circumference of the insertion portion 4 and the inner circumference of the insertion portion 511 of the sheath 509.

After that, the water supply port 513 and the water supply/suction unit 529 are connected by the water supply tube 531, and moreover, the suction port 514 and the insufflator 530 are connected by the exhaust tube 533, and then, the insertion portion 4 of the ultrasonic treatment instrument 501 is inserted into a body cavity and the tissue to be treated in the body cavity is treated.

The following operation is performed by a control signal sent from control means 570, which is a control portion disposed in the ultrasonic generator 528 of the ultrasonic treatment apparatus 500 to the water supply/suction unit 529 and the insufflator 530 through the communication cable 538. That is, it is performed by operation control of the control means 570.

When treating a tissue to be treated in a body cavity, after the tissue to be treated is gripped by the grip portion 5, as shown in FIG. 33, first at Step S21, the ultrasonic vibration switch 535 as an ultrasonic vibration switch is turned on, the vibrator 7 receives power supply from the ultrasonic generator 528 and performs ultrasonic driving.

The vibration of the vibrator 7 is transmitted to the grip portion 5 through the probe 8. By this, the tissue to be treated is heated by friction against the tissue to be treated by the vibration of the probe 8 at the tip end side, and treatment such as coagulation ablation is performed.

Then, at Step S22, it is determined by operation of the water supply/suction unit 529 whether a fluid of water is being supplied to the grip portion 5 and the probe 8 from the tip end opening portion 543.

If the fluid of water is being supplied from the tip end opening portion 543, on one hand, the routine branches to Step S23, where the water supply/suction unit 529 is stopped and the water supply from the tip end opening portion 543 is stopped. And after it is reconfirmed at the subsequent Step S30 that the ultrasonic vibration switch 535 is on, the routine goes onto Step S24. On the other hand, at Step S22, if the fluid of water is not supplied from the tip end opening portion 543, the routine goes onto Step S24.

At Step S24, in conjunction with heating treatment of the tissue to be treated by the grip portion 5, the insufflator 530 is operated, and mist or smoke generated in the treatment is sucked. Specifically, the fluid sucked by the insufflator 530 advances into the tip end flow passage 526 from the tip end opening portion 543 and then, flows through the flow passages 512, 5101, 514r and is discharged from the insufflator 530 through the exhaust tube 533 connected to the suction port 514.

When the treatment of the tissue to be treated by the grip portion 5 is finished, at Step S25, the switch 535 is turned off, the power supply from the ultrasonic generator 528 is shut off, and the vibrator 7 is stopped.

Then, in conjunction with the stop of the vibrator 7, at Step S26, the insufflator 530 is stopped and the suction of the mist or smoke generated in the treatment is stopped. In this case, the stop of the insufflator 530 may be delayed than the stop of the vibrator 7, not in synchronization with the stop of the vibrator 7. After that, the routine goes onto Step S27.

At Step S27, the water supply/suction unit 529 is operated for preset time of several seconds, and the fluid is supplied from the tip end opening portion 543 to the grip portion 5 or the probe 8.

Specifically, the fluid supplied from the water supply/suction unit 529 advances into the flow passage 513r of the water supply port 513 through the water supply tube 531 and then, flows through the flow passage 512 of the insertion portion 511 of the sheath 509 and is supplied to the grip portion 5 or the probe 8 from the tip end opening portion 543 of the tip end flow passage 526 as mentioned above in the state where the flow velocity is increased by the venturi effect.

By this, the grip portion 5 or the probe 8 having been heated by the treatment is cooled. Moreover, stain on the grip portion 5 and the probe 8 having adhered by the treatment is removed by the water supply of the fluid.

Next, at Step S28, it is determined whether the time during which the fluid is supplied from the tip end opening portion 543 has reached the preset several seconds. If the preset several seconds have not elapsed, the routine returns to Step S22 and at the subsequent Step S23, after the time to supply the fluid of water or air has passed for several seconds, the water supply/suction unit 529 is stopped. And at the subsequent Step S30, the off state of the switch 535 is confirmed, and then, the routine is finished.

Returning to Step S28, if the time to supply the fluid of water from the tip end opening portion 543 has passed for the preset several seconds, the routine goes onto Step S29, and the supply of the fluid of water from the tip end opening portion 543 is stopped by the stop of the water supply/suction unit 529, and then, the routine is finished.

In this way, in the ultrasonic treatment instrument 501 of this embodiment, it is shown that the flow passages 510r, 512, 513r, 526 are provided for supplying a fluid of water to the ultrasonic treatment instrument 501, and the flow passages 5101, 512, 514r, 526 are provided for sucking the fluid from within the body cavity in a treatment. Moreover, it is shown that the flow passage 512 provided between the insertion portion 4 and the insertion portion 511 serves as a flow passage for supplying a fluid of water and also as a flow passage for sucking the fluid from within the body cavity. Furthermore, it is shown that the tip end opening portion 543 of the flow passages 510r, 5101, 512, 513r, 514r, 526 is provided at the tip end side of the ultrasonic treatment instrument 501 so that it is located on the vicinity side of the probe 8.

According to this construction, the smoke or mist generated in the ultrasonic treatment is effectively sucked by the insufflator 530 through the flow passages 5101, 512, 514r, 526, and the treatment using the treatment instrument can be surely performed while ensuring the visual field of an operator all the time.

Moreover, after the ultrasonic treatment, a fluid whose flow velocity has increased by the venturi effect can be supplied by the water supply/suction unit 529 through the flow passages 510r, 512, 513r, 526 from the opening portion 543 to the grip portion 5 and the probe 8 whose temperature has become high due to frictional heat, and the grip portion 5 and the probe 8 which are heated after the treatment can be cooled in a shorter time with a smaller water supply amount than the case where the sectional area A2 of the tip end opening portion 543 is not narrowed.

Furthermore, since the tip end opening portion 543 is provided in the vicinity of the probe 8 and the tip end opening portion 543 is located on the probe 8 side due to the engagement between the engaging member 544 and the engagement receiving portion 545, in whichever direction the probe 8 is oriented, the fluid can be surely supplied to the probe 8. And the probe 8, which is a heat generating source, can be surely cooled.

From the above, the ultrasonic treatment instrument 501 whose convenience is improved for an operator can be provided. Other effects are the same as those of the ultrasonic treatment instrument 1 in the above-mentioned first embodiment.

A variation will be shown below. According to the construction of the ultrasonic treatment apparatus 500 of this embodiment, at the ultrasonic output, not only that smoke or mist is sucked but also a high frequency can be supplied from the high frequency power supply 527 connected to the insufflator 530 to the ultrasonic treatment instrument 501 and the ultrasonic suction treatment instrument 532 by the communication cable 538. That is, even while a high-frequency output is carried out, the smoke and mist can be sucked by interlocking operation of the insufflator 530 as with the ultrasonic output.

Also, the fluid of water supplied from the tip end opening portion 543 is shown to be used to cool the grip portion 5 and the probe 8 after a treatment. Not limited to this, the fluid may be used for washing inside a body cavity by supplying a fluid of water into the body cavity in this embodiment.

Furthermore, the ultrasonic treatment instrument 501 to be inserted into the sheath 509 may be a high-frequency treatment instrument, not shown, which is connectable to the high frequency power supply 527, and even in this case, as with this embodiment, supply and suction of a fluid of water are possible.

### Sixth Embodiment

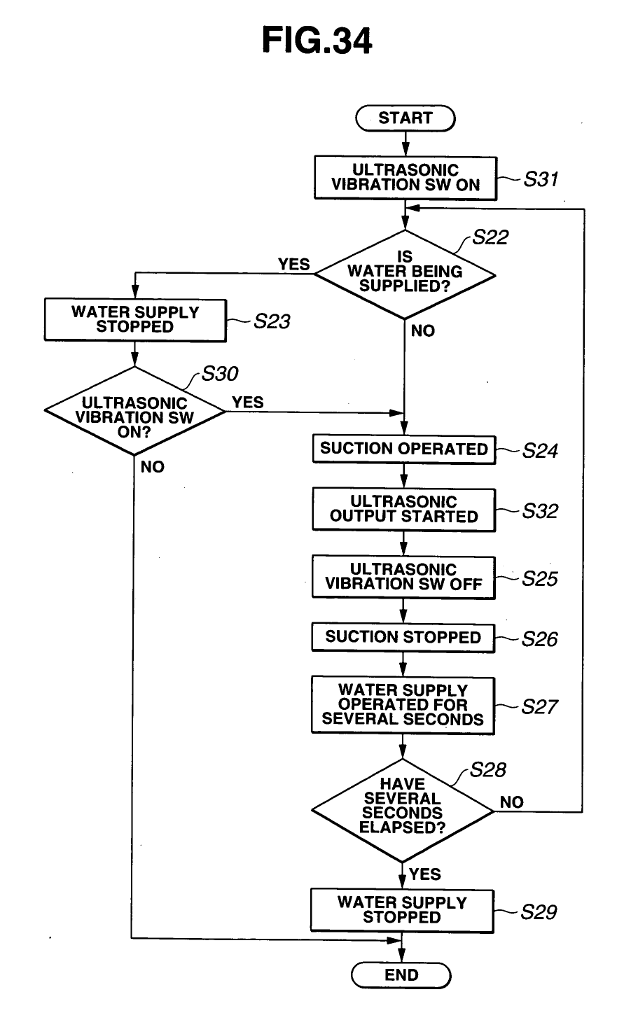

FIG. 34 is a flowchart showing a control method of the ultrasonic treatment apparatus having the ultrasonic treatment instrument showing a sixth embodiment of the present invention.

The control method of the ultrasonic treatment apparatus having the ultrasonic treatment instrument in this embodiment is different from the fifth embodiment shown in FIG. 33 in the point that suction by the insufflator 530 is performed before heating treatment of a tissue to be treated by the grip portion 5. The construction of the ultrasonic treatment instrument, the ultrasonic treatment apparatus is the same as that of the fifth embodiment. Therefore, only the difference will be described and the same reference numerals are given to the same construction in the fifth embodiment, whose description will be omitted.

As shown in FIG. 34, first at Step S31, the ultrasonic vibration switch 535 is turned on and a sound signal is inputted. Then, at Step S22, by operation of the water supply/suction unit 529, it is determined whether a fluid of water is being supplied from the tip end opening portion 543 to the grip portion 5 and the probe 8.

When a fluid of water is being supplied from the tip end opening portion 543, the routine branches to Step S23, where supply of the fluid of water from the opening portion 543 is stopped by stopping the water supply/suction unit 529 and at the subsequent Step S30, it is reconfirmed that the switch 535 is on, and then, the routine moves onto Step S24. If, at step S22, the fluid of water is not supplied from the tip end opening portion 543, the routine goes onto Step S24.

At Step S24, a control signal is sent by the ultrasonic generator 528 through the communication cable 538 to the insufflator 530, and suction of smoke or the like is started by the insufflator 530. At the subsequent Step S32, a control signal to start suction is sent from the insufflator 530 through the communication cable 538 to the ultrasonic generator 528, and upon receiving it, electric power is supplied from the ultrasonic generator 528 to the vibrator 7 so that ultrasonic output is started. This ultrasonic output may be started after a certain time has elapsed since start of the suction. After that, the routine goes onto Step S25.

Since Steps S25 to S29 are the same as Steps S25 to S29 of the above-mentioned fifth embodiment, the explanation will be omitted.

According to this embodiment, by operating suction by the insufflator 530 prior to heating treatment of a tissue to be treated using ultrasonic wave, mist and smoke in a treatment through ultrasonic output are surely sucked, and the ultrasonic treatment instrument 501 which can ensure the visual field of an operator favorably and whose convenience is good can be provided. The other effects are the same as those of the above-mentioned fifth embodiment.

### Seventh Embodiment

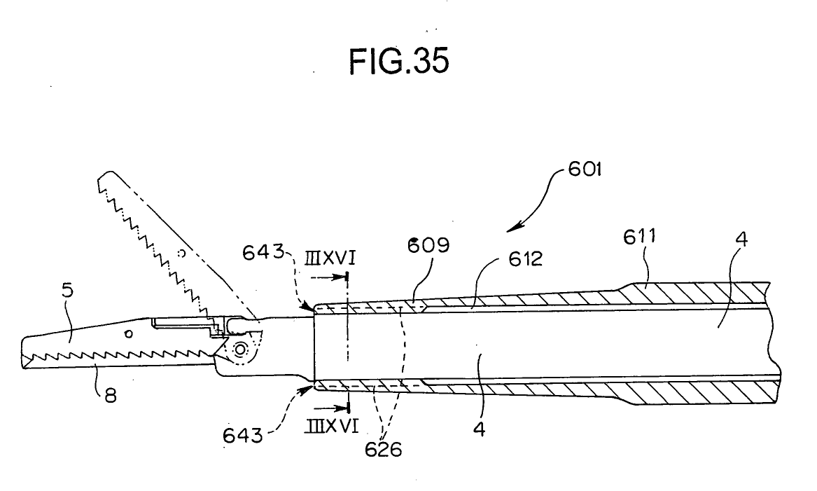

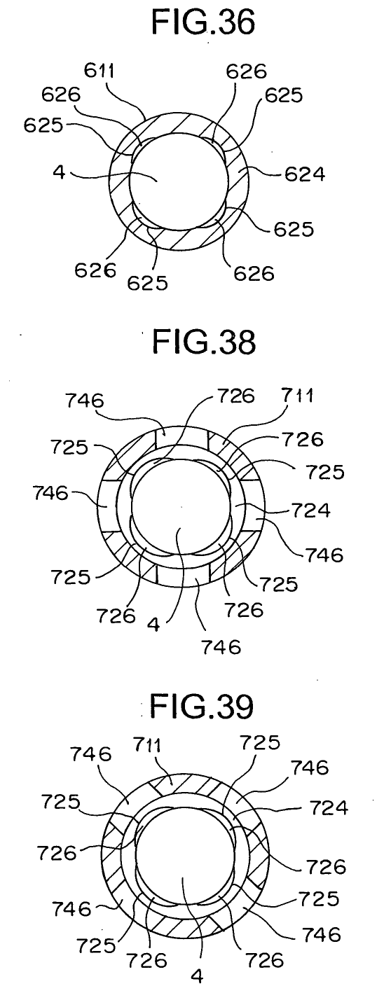

FIG. 35 is an enlarged sectional view of a tip end side of a sheath of an ultrasonic treatment instrument showing a seventh embodiment of the present invention, and FIG. 36 is a sectional view along IIIXVI-IIIXVI line in FIG. 35.

The construction of the ultrasonic treatment instrument of this embodiment is different from the fifth embodiment shown in FIGS. 27 to 33 in the point that a plurality of tip end opening portions are provided. Therefore, only the difference will be described and the same reference numerals are given to the same construction in the fifth embodiment, whose description will be omitted.

As shown in FIG. 35, when a sheath 609, which is a cylindrical member having the same construction as that of the sheath 509 of the fifth embodiment, is attached to the insertion portion 4 of an ultrasonic treatment instrument 601, the tip end of the sheath 609 is located nearer to the base end side than the grip portion 5 so that the tip end sides of the grip portion 5 and the probe 8 are exposed.

As shown in FIG. 36, a support portion 624 brought into contact with the outer circumference of the insertion portion 4 is formed on an inner circumference on the tip end portion side of an insertion portion 611, and an arc 625 is formed on the inner circumference of the tip end portion of the insertion portion 611 at four locations, for example. The number of locations to form the arc 625 may be any, as long as it is plural.

Four tip end flow passages 626 are formed between the four arcs 625 and the outer circumference of the insertion portion 4, and the tip end flow passages 626 communicate to a flow passage 612 formed between the inner circumference of the insertion portion 611 and the outer circumference of the insertion portion 4.

Four tip end opening portions 643, which are openings on the tip end side of the four tip end flow passages 626, are formed between the four arcs 625 and the outer circumference of the insertion portion 4. A total of sectional areas of the four tip end opening portions 643 in this embodiment is substantially equal to the sectional area of the tip end opening portion 543 of the fifth embodiment. The four tip end opening portions 643 are opened in the vicinity of the probe 8 from the grip portion 5 opposite to the grip portion 5 and the probe 8 and located at positions in the vicinity of the tip end side of the probe 8 from the grip portion 5. That is, the four tip end opening portions 643 are opened at least toward the tip end side of the probe 8.

In the ultrasonic treatment instrument 601 of this embodiment, the engaging member 544 provided at the tip end of the rotating knob 503 of the ultrasonic treatment instrument 501 and the engagement receiving portion 545 provided at the base end portion 519 of the connection portion 515 of the sheath 509 in the above-mentioned fifth embodiment are not disposed.

According to the construction of the ultrasonic treatment instrument 601 of this embodiment, since the tip end opening portions 643 are provided in plural, there is no need to position the tip end opening portions 643 with respect to the probe 8 as in the above-mentioned fifth embodiment. And in whichever direction in the sheath 609 the insertion portion 4 is inserted, water can be supplied to the probe 8 and the grip portion 5 when a fluid of water is supplied from the water supply/suction unit 529. Therefore, the probe 8 and the grip portion 5 can be surely cooled. The other effects are the same as those of the above-mentioned fifth embodiment.

### Eighth Embodiment

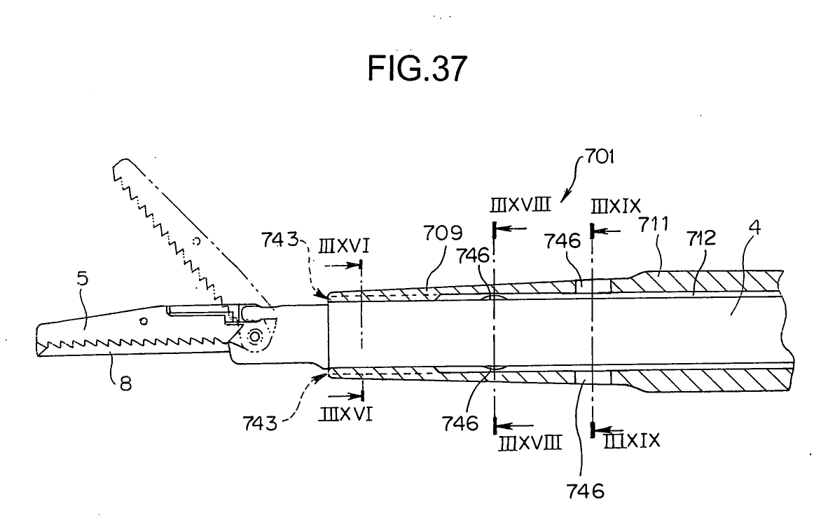

FIG. 37 is an enlarged sectional view of a tip end side of an ultrasonic treatment instrument showing an eighth embodiment of the present invention, FIG. 38 is a sectional view along IIIXVIII-IIIXVIII line in FIG. 37, and FIG. 39 is a sectional view along IIIXIX-IIIXIX line in FIG. 37.

The construction of the ultrasonic treatment instrument of this embodiment is different from the fifth embodiment shown in FIGS. 27 to 33 in the point that a side opening portion is provided to the insertion portion of the sheath other than the tip end opening portions. Therefore, only the difference will be described and the same reference numerals are given to the same construction in the fifth embodiment, whose description will be omitted.

As shown in FIG. 37, when a sheath 709, which is a cylindrical member having the same construction as that of the sheath 509 of the fifth embodiment is attached to the insertion portion 4 of an ultrasonic treatment instrument 701, the tip end of the sheath 709 is located nearer to the base end side than the grip portion 5 so that the tip end sides of the grip portion 5 and the probe 8 are exposed.

Also, as shown in FIGS. 38, 39, a support portion 724 in contact with the outer circumference of the insertion portion 4 is formed on an inner circumference on the tip end side of the insertion portion 711, and a plurality of, four arcs 725, for example, are formed on the inner circumference on the tip end side of the insertion portion 711.

Four tip end flow passages 726 are formed between the four arcs 725 and the outer circumference of the insertion portion 4, and the tip end flow passages 726 communicate to a flow passage 712 formed between the inner circumference of the insertion portion 711 and the outer circumference of the insertion portion 4.

Moreover, at the tip end side of the insertion portion 711 of the sheath 709, side opening portions 746 are provided at four locations at every 90 degrees on the circumference as shown in FIG. 38, and furthermore, as shown in FIG. 39, side opening portions 746 are also provided at four locations on the base end side of the sheath 709 at positions offset by 45 degrees, respectively, on the circumference from the positions shown in FIG. 38.

According to the construction of the ultrasonic treatment instrument 701 in this way, when sucking mist or smoke by the insufflator 530 from tip end opening portions 743 opened at least opposite to the tip end side of the probe 8 in the vicinity of the probe 8 from the grip portion 5 and located at positions in the vicinity of the tip end side of the probe 8 from the grip portion 5, even if the tip end opening portions 743 are blocked by a tissue or blood, suction can be performed from the side opening portions 746. The other effects are the same as those of the above-mentioned fifth embodiment.

### Ninth Embodiment

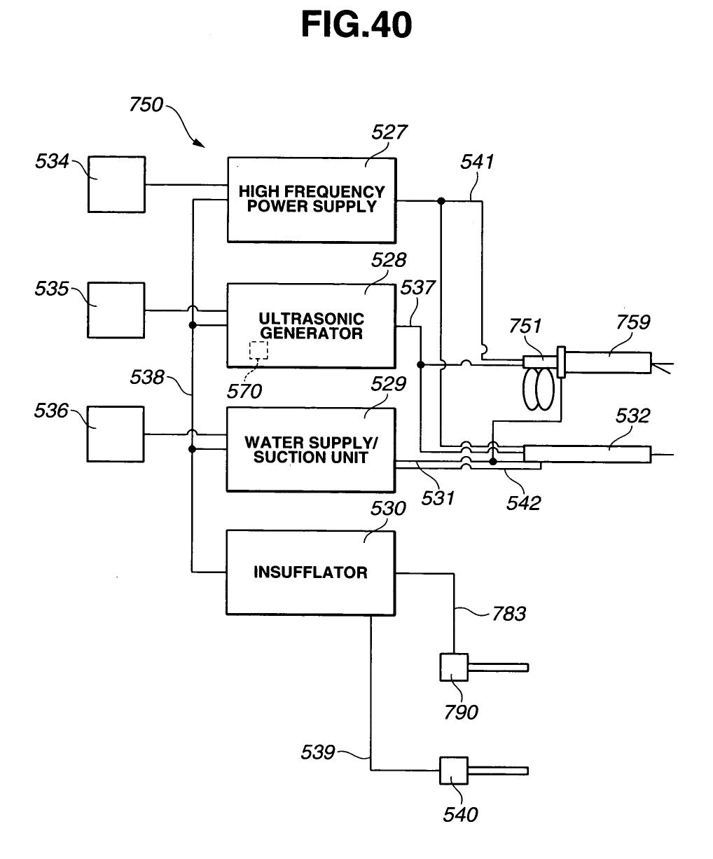

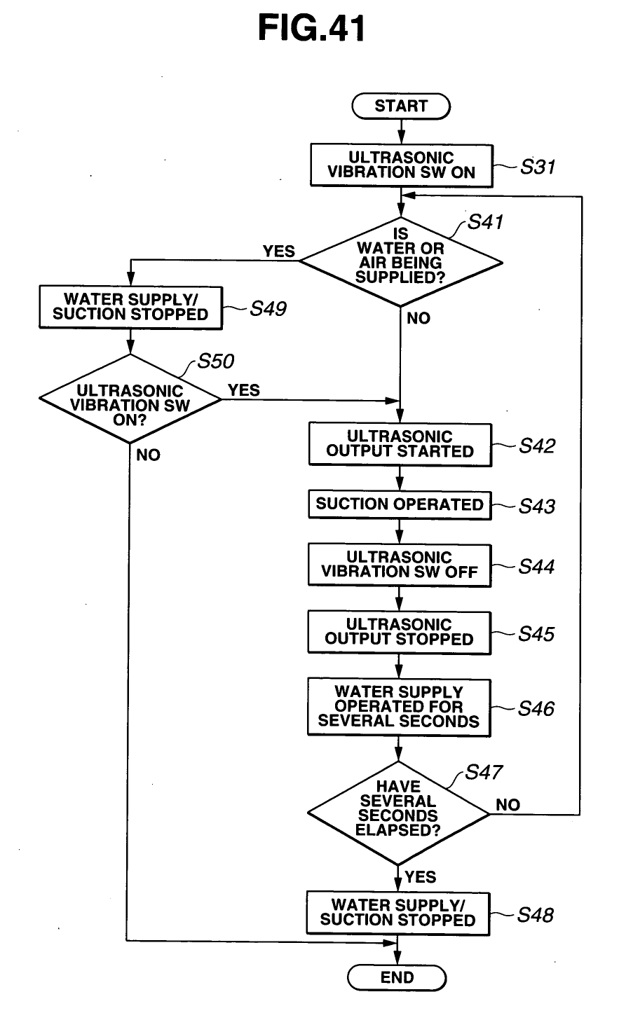

FIG. 40 is a block diagram schematically showing a construction of an ultrasonic treatment apparatus having an ultrasonic treatment instrument showing a ninth embodiment of the present invention and FIG. 41 is a flowchart showing a control method of an ultrasonic treatment apparatus in FIG. 40.

A control method of an ultrasonic treatment apparatus having an ultrasonic treatment instrument of this embodiment is different from the fifth embodiment shown in FIG. 33 in the point that not only water supply but suction by the insufflator 530 is performed even after heating treatment of a tissue to be treated by the grip portion 5. The construction of the ultrasonic treatment instrument, the ultrasonic treatment apparatus is the same as that of the fifth embodiment. Therefore, only the difference will be described and the same reference numerals are given to the same construction in the fifth embodiment, whose description will be omitted.

As shown in FIG. 40, another trocar 790 for suction which is different from the trocar 540 is connected to the insufflator 530 of an ultrasonic treatment apparatus 750 through an exhaust tube 783. In this embodiment, a sheath 759 does not have to have a suction port.

As shown in FIG. 41, first at Step S31, the ultrasonic vibration switch 535 is turned on and a signal is inputted. Then, at Step S41, by operation of the water supply/suction unit 529, it is determined whether a fluid of water is being supplied from the tip end opening portion to the grip portion 5 and the probe 8, and by operation of the insufflator 530, it is determined whether smoke, mist or the like is sucked from the trocar 790.

If the fluid of water is supplied from the tip end opening portion 543 and the smoke, mist or the like is sucked by the trocar 790, the routine branches to Step S49, where the water supply/suction unit 529, the insufflator 530 are stopped, supply of the fluid of water from the opening portion is stopped, and suction of the smoke, mist or the like by the trocar 790 is stopped. And at the subsequent Step S50, it is reconfirmed that the switch 535 is on and then, the routine goes onto Step S42. At Step S41, if the fluid is not supplied from the tip end opening portion and the smoke, mist or the like is not sucked by the trocar 790, the routine goes onto Step S42.

At Step S42, upon receiving electric power from the ultrasonic generator 528, the vibrator 7 performs ultrasonic driving. By this, a tissue to be treated is heated by friction with the grip portion 5 by vibration of the probe 8 on the tip end side of the ultrasonic treatment instrument 751, and treatment such as coagulation ablation is carried out.

At the subsequent Step S43, a control signal is sent from the ultrasonic generator 528 through the communication cable 538 to the insufflator 530, and the insufflator 530 starts suction by the trocar 790. The suction is performed substantially at the same time with ultrasonic driving of the vibrator 7.

When the treatment of the tissue to be treated by the grip portion 5 is finished, the switch 535 is turned off at Step S44, and at the subsequent Step S45, power supply from the ultrasonic generator 528 is shut off, and the vibrator 7 is stopped.

At Step S46, the water supply/suction unit 529 and the insufflator 530 are operated for a preset time of several seconds and a fluid of water is supplied from the tip end opening portion to the grip portion 5 or the probe 8, while smoke, mist or the like is sucked from the trocar 790.

Then, at Step S47, it is determined whether the time during which the fluid is supplied and sucked has reached the preset several seconds or not. If the preset several seconds have not elapsed, the routine returns to Step S41 and at the subsequent Step S49, supply and suction of the fluid are performed for several seconds. And the water supply/suction unit 529, the insufflator 530 are stopped, and at the subsequent Step S50, the off state of the switch 535 is confirmed and then, the routine is finished.

Returning to Step S47, if the time during which the fluid of water is supplied/sucked has reached the preset several seconds, the routine goes onto Step S48, where the water supply/suction unit 529 and the insufflator 530 are stopped and then, the routine is finished.

In this way, according to this embodiment, since the suction operation can be prolonged after an ultrasonic treatment, mist or smoke can be sucked for a longer time. Thus, the visual field of an operator can be favorably kept. The other effects are the same as those of the above-mentioned fifth embodiment.

### Tenth Embodiment

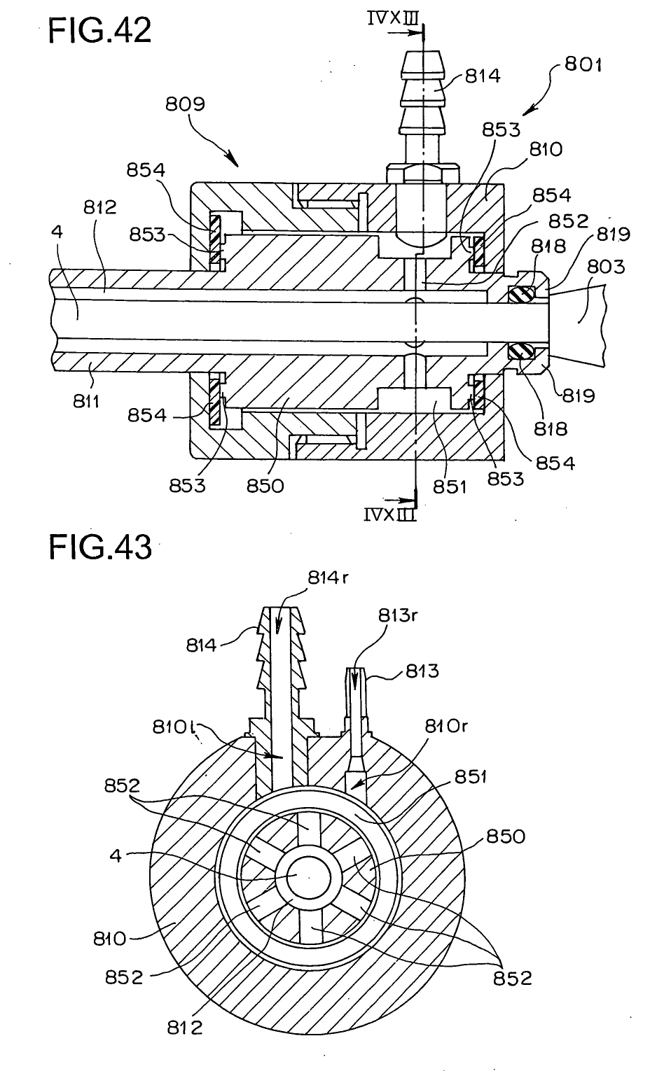

FIG. 42 is an enlarged sectional view of a base end side of an insertion portion of an ultrasonic treatment instrument showing a tenth embodiment of the present invention and FIG. 43 is a sectional view along IVXIII-IVXIII line in FIG. 42.

The construction of an ultrasonic treatment instrument of this embodiment is different from the fifth embodiment shown in FIGS. 27 to 33 in the point that the ultrasonic treatment instrument has a structure to prevent tangling of a water supply tube connected to a water supply port and an exhaust tube connected to a suction port when rotating the rotating knob. Therefore, only the difference will be described and the same reference numerals are given to the same construction in the fifth embodiment, whose description will be omitted.

As shown in FIG. 42, a sheath 809, which is a cylindrical member of an ultrasonic treatment instrument 801 of this embodiment, has its major part comprised by a body portion 810 and a rotating portion 850 provided inside the body portion 810.

Projection portions 853 are formed respectively on the tip end side and the base end side of the rotating portion 850, and the projection portions 853 are in contact only at the apexes with elastic plates 854 disposed respectively on the tip end side and the base end side between the body portion 810 and the rotating portion 850.

An inner diameter of a hole of an insertion portion 811 is formed larger than the insertion portion 4. By this, a flow passage 812 is formed between an inner circumference of the insertion portion 811 and the outer circumference of the insertion portion 4. Also, a plurality of holes 852 are formed along the circumferential direction on the rotating portion 850, and the plurality of holes 852 communicate to a space 851 between the inner circumference of the body portion 810 and the outer circumference of the rotating portion 850. Therefore, the space 851 and the flow passage 812 communicate to each other.

Furthermore, as shown in FIG. 43, a water supply port 813, an inlet to be connected to the water supply/suction unit 529, is provided to the body portion 810. To the water supply port 813, the water supply tube 531 to be connected to the water supply/suction unit 529 is connected.

The water supply port 813 has a flow passage 813r, and the flow passage 813r communicates to a flow passage 810r of the body portion 810. Furthermore, the flow passage 813r communicates to the space 851, by which the flow passage 813r communicates to the flow passage 812.

Moreover, a suction port 814, which is an outlet to be connected to the insufflator 530 having a scavenging function, is provided to the body portion 810. To the suction port 814, the exhaust tube 533 to be connected to the insufflator 530 is connected.

The suction port 814 has a flow passage 814r, and the flow passage 814r communicates to a flow passage 8101 of the body portion 810. Moreover, the flow passage 814r communicates to the space 851, by which the flow passage 814r communicates to the flow passage 812.

Moreover, the water supply port 813, the suction port 814 constitute an inlet/outlet for performing at least one of supply to the flow passage 812 of a fluid supplied to the treatment portion comprising the tip end sides of the grip portion 5 and the probe 8 and discharge from the flow passage 812 of the fluid sucked from within the body cavity.

At a base end portion 819 of the rotating portion 850, a recess portion opened toward the base end side is provided, and an elastic member 818 made of an O-ring, for example, is fitted in the recess portion. An inner diameter of a hole of the O-ring of the elastic member 818 is formed smaller than the outer diameter of the insertion portion 4 to such an extent that the insertion portion 4 can be inserted, and when the insertion portion 4 is inserted into the hole of the O-ring of the elastic member 818, the elastic member 818 is elastically deformed between the recess portion and the insertion portion 4, and air tightness is kept between the insertion portion 4 and the elastic member 818. That is, it is brought into close contact with the outer circumference of the insertion portion 4 in the deformed state. Also, an inner circumference of an opening of the recess portion is formed larger than the outer diameter of the insertion portion 4 and smaller than a diameter on the tip end side of a rotating knob 803.

The rotating portion 850 of the ultrasonic treatment instrument 801 having this construction is in contact with the elastic plate 854 only at the apexes of the projection portions 853, and the other outer circumferential portion of the rotating portion 850 is not in contact. Therefore, when the rotating knob 803 is rotated, the rotating portion 850 in conjunction with the insertion portion 4 is rotated with respect to the body portion 810. That is, only the rotating portion 850 is rotated separately from the body portion 810.

By this, when the rotating knob 803 is rotated, tangling of the water supply tube 531 connected to the water supply port 813 and the exhaust tube 533 connected to the suction port 814 is prevented. The other effects are the same as those of the above-mentioned fifth embodiment.

### Eleventh Embodiment

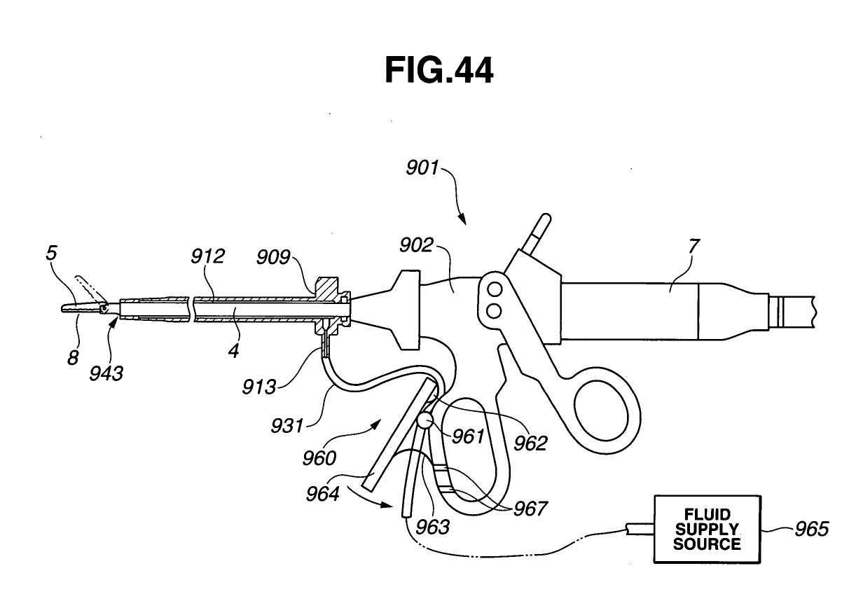

FIG. 44 is a view showing an ultrasonic treatment instrument with only a part of an insertion portion in section showing an eleventh embodiment of the present invention.

The construction of an ultrasonic treatment instrument of this embodiment is different from the fifth embodiment shown in FIGS. 27 to 33 in the point that the ultrasonic treatment instrument has a structure in which supply of a fluid of water can be carried out with good operability. Therefore, only the difference will be described and the same reference numerals are given to the same construction in the fifth embodiment, whose description will be omitted.

As shown n FIG. 44, a sheath 909, which is a cylindrical member, is attached to the insertion portion 4 of an ultrasonic treatment instrument 901, and a water supply port 913 having an inlet is formed to the sheath 909. The water supply port 913 has a detachable water supply tube 931, and the water supply tube 931 is connected to a fluid supply source 965, which is a fluid supply portion. In this case, the water supply tube 931 may be connected to the water supply/suction unit 529.

A lever 964, which is switching means of a pinch valve 960, is mounted on a fixed handle 902, which is an operation portion, on the base end side of the insertion portion 4 through a mounting portion 967, which is mounting means, and a projection portion 962 capable of contact with a part of a water supply tube 931 is provided to the lever 964. The pinch valve 960 may be detachable with respect to the fixed handle 902 in the mounting portion 967.

The projection portion 962 presses a part of the water supply tube 931 when a fluid of water is not supplied from a tip end opening portion 943 and crushes a part of the water supply tube 931 so that the fluid does not pass through the water supply tube 931.

When water is to be supplied, one end side of the lever 964 is rotated in a direction of an arrow in FIG. 44, and the lever 964 is rotated around a fulcrum 961 and the projection portion 962 is rotated in a direction away from the water supply tube 931. By this, a flow passage of the water supply tube 931 is formed and a fluid of water is supplied from the tip end opening portion 943 opened in the vicinity of the probe 8 from the grip portion 5 at least opposite to the tip end side of the probe 8, and located in the vicinity position on the tip end side of the probe 8 from the grip portion 5.

In order to stop the supply of the fluid of water, by releasing manipulation of the lever 964 by an operator, the lever 964 returns by an action of a spring 963 to an original position where a part of the water supply tube 931 is crushed.

According to this construction, water supply operation of a fluid from the tip end opening portion 943 through the flow passage 912 can be performed at a timing intended by an operator and with good operability during a treatment. The other effects are the same as those of the above-mentioned fifth embodiment.

[Appendage]

As mentioned above in detail, according to the embodiments of the present invention, the following constructions can be obtained. That is,
(1) A medical treatment instrument comprising an elongated insertion portion to be inserted into a body cavity;

a treatment portion provided at a tip end of the insertion portion for treating a tissue to be treated in the body cavity by heating the tissue;

a cylindrical member into which at least a part of the insertion portion is inserted;

a flow passage formed between an outer circumference of the insertion portion and an inner circumference of the cylindrical member and through which at least one of a fluid supplied to the treatment portion and the fluid sucked from within the body cavity flows;

an inlet/outlet communicating with the flow passage for performing at least one of supply to the flow passage of the fluid supplied to the treatment portion and discharge from the flow passage of the fluid sucked from within the body cavity; and

an opening portion communicating with the flow passage and opened opposite to the treatment portion.
(2) A medical treatment instrument comprising an elongated insertion portion to be inserted into a body cavity;

a treatment portion provided at a tip end of the insertion portion for treating a tissue to be treated in the body cavity by applying heat to the tissue;

a hollow and lengthy cylindrical member covering the insertion portion from a near side to a tip end side thereof and arranging the treatment portion in the state projected outside the tip end side;

a flow passage formed by a space between the insertion portion inserted into the cylindrical member and an inner circumferential face of the cylindrical member and capable of passage of a fluid from the near side to the tip end side of the cylindrical member;

a base for fluid supply communicating with the flow passage, provided at the near side of the cylindrical member and connected to a fluid supply source for supplying the fluid supplied to the tip end side of the cylindrical member through the flow passage;

a base for suction communicating with the flow passage, provided at the near side of the cylindrical member and connected to a suction source for sucking the fluid from the tip end side of the cylindrical member through the flow passage; and

an opening portion capable of supply of a fluid to the treatment portion arranged in the state projected from the tip end of the cylindrical member through the flow passage from the base for fluid supply and provided at the tip end portion of the cylindrical member for sucking the fluid from the vicinity of the treatment portion from the base for suction through the flow passage.
(3) The medical treatment instrument described in appendage 1 or 2, wherein the cylindrical member has a diameter of the inner circumferential face formed larger than that of the insertion portion inserted into the cylindrical member, and the treatment portion provided at the tip end of the insertion portion is projected from the cylindrical member.
(4) A medical treatment instrument comprising a cylindrical member detachably mounted on an insertion portion of a medical treatment instrument having a treatment portion for performing a treatment using heat and the elongated lengthy insertion portion to be inserted into a body cavity and covering the insertion portion, the cylindrical member being provided with a channel through which the insertion portion is passed, an opening portion provided in the vicinity of the treatment portion, and a water supply/suction tool provided with a flow passage for water supply/suction and a base at the flow passage to be connected to a water supply source/suction source.
(5) The medical treatment instrument described in appendage 4, wherein the channel has a shape and dimensions such that the channel is not in close contact with the insertion portion over the whole circumference and the flow passage is formed by a space between the channel and the insertion portion in the state assembled to the insertion portion.
(6) A water supply/suction control system for a medical treatment instrument in which control of the water supply/suction is conducted by detecting an output of the medical treatment instrument described in appendages 1 to 5.
(7) A water supply/suction system for the medical treatment instrument described in appendage 6, wherein the medical treatment instrument is an ultrasonic coagulation ablation instrument.
(8) The water supply/suction system for a medical treatment instrument described in appendage 6, wherein the medical treatment instrument is a high-frequency treatment instrument.
(9) The water supply/suction system for a medical treatment instrument described in appendage 6, wherein the medical treatment instrument is a laser treatment instrument.
(10) A water supply/suction system for a medical treatment instrument comprising a medical treatment instrument having a treatment portion for performing a treatment using heat and an elongated lengthy insertion portion to be inserted into a body cavity, an opening portion in the vicinity of the treatment portion, a flow passage for water supply/suction to/from the treatment portion in the insertion portion, and a base to be connected to a water supply source/suction source to/from the flow passage provided at a base end portion of the insertion portion.
(11) The water supply/suction system for a medical treatment instrument described in appendage 10, wherein a device is provided to the treatment portion for controlling a timing and a duration in performing water supply/suction.
(12) The water supply/suction system for a medical treatment instrument described in appendage 10, comprising a heating portion constituting the treatment portion;

a grip portion constituting the treatment portion for opening/closing with respect to the heating portion; and

an elongated drive shaft connected to the grip portion for opening/closing operation, and

wherein a channel into which the drive shaft is passed has a shape and dimensions such that the channel is not in close contact with the insertion portion over the whole circumference, and the flow passage is formed in a space between the channel and the insertion portion in the state assembled to the insertion portion.
(13) The water supply/suction system for a medical treatment instrument described in appendage 10, wherein an elongated heating portion with at least a tip end side constituting the treatment portion is provided,

a channel into which the heating portion is passed has a shape and dimensions such that the channel is not in close contact with the insertion portion over the whole circumference, and the flow passage is formed in a space between the channel and the insertion portion in the state assembled to the insertion portion.
(14) The water supply/suction system for a medical treatment instrument described in appendage 10, wherein control of the water supply/suction is conducted by detecting an output of the medical treatment instrument.
(15) The water supply/suction system for a medical treatment instrument described in appendage 10, wherein the medical treatment instrument is an ultrasonic coagulation ablation instrument.
(16) The water supply/suction system for a medical treatment instrument described in appendage 10, wherein the medical treatment instrument is a high-frequency treatment instrument.
(17) The water supply/suction system for a medical treatment instrument described in appendage 10, wherein the medical treatment instrument is a laser treatment instrument.
(18) A medical treatment instrument having a treatment portion for a treatment using heat and an elongated lengthy insertion portion to be inserted into a body cavity, in which a shape is provided for a heat sink at the treatment portion.
(19) A cooling device for a medical treatment instrument having a treatment portion for a treatment using heat and an elongated lengthy insertion portion to be inserted into a body cavity, comprising a cooling member for absorbing the heat of the treatment portion and the cooling device holding the cooling member and movable along the insertion portion, wherein the treatment portion is cooled by operating the cooling device after the treatment so as to bring the cooling member into contact with the treatment portion.
(20) The cooling device for a medical treatment instrument described in appendage 19, wherein the cooling member is a cloth member immersed in a normal saline.
(21) The cooling device for a medical treatment instrument described in appendage 19, wherein the cooling member is made of a biocompatible material.
(22) The medical treatment instrument described in appendage 1, wherein a sectional area of the opening portion is smaller than a sectional area of the flow passage.
(23) The medical treatment instrument described in appendage 1 or 22, wherein the treatment portion is comprised by a heating portion for heating the tissue to be treated and an opening/closing portion capable of opening/closing with respect to the heating portion, and the opening portion is opened at least opposite to the heating portion.
(24) The medical treatment instrument described in appendage 22, wherein the sectional area of the opening portion is not more than 30% of the sectional area of the flow passage.
(25) The medical treatment instrument described in appendage 23, comprising an operating knob provided at a base end side of the insertion portion for rotating the insertion portion in the axial direction, and

engagement means for rotatably engaging the operating knob with the cylindrical member together with the insertion portion by rotating operation of the operating knob.
(26) The medical treatment instrument described in appendage 23, wherein the heating portion is a probe for transmitting ultrasonic vibration.
(27) A medical treatment instrument comprising:

an elongated insertion portion to be inserted into a body cavity;

a treatment portion provided at a tip end of the insertion portion and performing a treatment by heating a tissue to be treated in the body cavity when an electric energy is supplied from a power supply;

a cylindrical member into which at least a part of the insertion portion is inserted;

a flow passage formed between an outer circumference of the insertion portion and an inner circumference of the cylindrical member and through which a fluid sucked from within the body cavity flows;

an outlet communicating with the flow passage for discharging the fluid sucked from within the body cavity from the flow passage;

an opening portion communicating with the flow passage and provided at a position in the vicinity of the treatment portion;

a suction portion connected to the outlet for sucking the fluid; and

a control portion for operating the suction portion for a predetermined time before the treatment portion heats the tissue to be treated by the electric energy supplied from the power supply.
(28) A medical treatment instrument comprising:

an elongated insertion portion to be inserted into a body cavity;

a treatment portion provided at a tip end of the insertion portion for performing a treatment by heating a tissue to be treated in the body cavity;

an operation portion provided at a base end of the insertion portion for operating the treatment portion;

a cylindrical member into which at least a part of the insertion portion is inserted;

a flow passage formed between an outer circumference of the insertion portion and an inner circumference of the cylindrical member and through which a fluid supplied to the treatment portion flows;

an inlet communicating with the flow passage and for making the fluid supplied to the treatment portion flow to the flow passage;

an opening portion communicating with the flow passage and provided at a position in the vicinity of the treatment portion;

a fluid supply portion for supplying a fluid to the inlet;

a switching portion provided between the inlet and the fluid supply portion for switching on/off of supply of the fluid; and

mounting means for making the control portion detachable with respect to the grip portion.

In the appendages 1 to 9, by attaching the water supply/suction sheath to the insertion portion of the medical treatment instrument, the water supply/suction flow passage is formed in a gap between the inner side of the water supply/suction sheath and the outer side of the insertion portion. By this, mist suction or supply of water/air is enabled by making the opening portion provided at the tip end side, the water supply/suction flow passage and the water supply/suction port provided to the water supply/suction sheath communicate to each other. Also, suction is performed at output and suction is further performed for several seconds when the output is stopped so that the output is detected and water supply/suction is controlled.

Moreover, in the appendages 10 to 15, the water supply/suction port is provided to the rotating knob of the ultrasonic treatment instrument, the water supply/suction port, the drive shaft channel, which is a pipeline through which the drive shaft for driving the grip portion passes and the opening portion provided at the tip end of the inner sheath are made to communicate to each other so that water supply/suction can be performed. Also, suction is performed at output and suction is further performed for several seconds when the output is stopped so that the output is detected and water supply/suction is controlled.

Furthermore, in the appendages 5, 7, 9 and 11, the generator of the ultrasonic treatment instrument, the water/air supply unit and the suction unit are made to be operated in conjunction. The water/air supply unit and the suction unit are connected to the water supply port by the tube.

Moreover, in the appendage 16, a surface area of the probe is increased by providing a groove on a lower face of the probe so that the temperature of the probe having been raised by the ultrasonic treatment can be discharged easily.

Furthermore, in the appendages 17 to 19, a cooling member is provided on the lower face of the probe, and the temperature of the probe having been raised by the ultrasonic treatment can be cooled down easily by bringing the cooling member into contact with the probe.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A medical treatment instrument comprising at least:
an elongated insertion portion to be inserted into a body cavity;
a treatment portion provided at a tip end of the insertion portion for treating a tissue to be treated in the body cavity by heating the tissue;
a cylindrical member into which at least a part of the insertion portion is inserted;
a flow passage formed between an outer circumference of the insertion portion and an inner circumference of the cylindrical member and through which at least one of a fluid supplied to the treatment portion and the fluid sucked from within the body cavity flows;
an inlet/outlet communicating with the flow passage for performing at least one of supply to the flow passage of the fluid supplied to the treatment portion and discharge from the flow passage of the fluid sucked from within the body cavity; and
an opening portion communicating with the flow passage and opened opposite to the treatment portion.

2. The medical treatment instrument according to claim 1, wherein the cylindrical member has a diameter of an inner circumferential face formed larger than a diameter of the insertion portion inserted into the cylindrical member, and the treatment portion provided at the tip end of the insertion portion is projected from the cylindrical member.

3. The medical treatment instrument according to claim 1, wherein a sectional area of the opening portion is smaller than a sectional area of the flow passage.

4. The medical treatment instrument according to claim 1, wherein the treatment portion includes a heating portion for heating the tissue to be treated and an opening/closing portion capable of opening/closing with respect to the heating portion, and
the opening portion is opened at least opposite to the heating portion.

5. The medical treatment instrument according to claim 3, wherein the treatment portion includes a heating portion for heating the tissue to be treated and an opening/closing portion capable of opening/closing with respect to the heating portion, and
the opening portion is opened at least opposite to the heating portion.

6. A water supply/suction system for a medical treatment instrument, comprising at least:
a medical treatment instrument having a treatment portion for performing a treatment using heat and an elongated lengthy insertion portion to be inserted into a body cavity,
an opening portion in the vicinity of the treatment portion,
a flow passage for water supply/suction to/from the treatment portion disposed in the insertion portion, and
a base in the flow passage to be connected to a water supply source/suction source at a base end portion of the insertion portion.

7. The water supply/suction system for a medical treatment instrument according to claim 6, wherein a device is provided to the treatment portion for controlling a timing and a duration when performing water supply/suction.

8. The water supply/suction system for a medical treatment instrument according to claim 6, comprising at least a heating portion constituting the treatment portion;
a grip portion constituting the treatment portion for opening/closing with respect to the heating portion; and
an elongated drive shaft connected to the grip portion for opening/closing operation, and
wherein a channel into which the drive shaft is passed has a shape and dimensions such that the channel is not in close contact with the insertion portion over the whole circumference, and the flow passage is formed in a space between the channel and the insertion portion in the state assembled to the insertion portion.

9. The water supply/suction system for a medical treatment instrument according to claim 6, wherein at least an elongated heating portion is provided with at least a tip end side constituting the treatment portion,
a channel into which the heating portion is passed has a shape and dimensions such that the channel is not in close contact with the insertion portion over the whole circumference, and the flow passage is formed in a space between the channel and the insertion portion in the state assembled to the insertion portion.

10. The water supply/suction system for a medical treatment instrument according to claim 6, wherein control of the water supply/suction is conducted by detecting an output of the medical treatment instrument.

11. The water supply/suction system for a medical treatment instrument according to claim 6, wherein the medical treatment instrument is an ultrasonic coagulation ablation instrument.

12. The water supply/suction system for a medical treatment instrument according to claim 6, wherein the medical treatment instrument is a high-frequency treatment instrument.

13. The water supply/suction system for a medical treatment instrument according to claim 6, wherein the medical treatment instrument is a laser treatment instrument.
